(19) United States
(12) Patent Application Publication
(10) Pub. No.: US 2024/0093318 A1
(43) Pub. Date: Mar. 21, 2024

(54) METHOD FOR DIAGNOSING RESPIRATORY PATHOGENS AND PREDICTING COVID-19 RELATED OUTCOMES

(71) Applicants: THE REGENTS OF THE UNIVERSITY OF COLORADO, A BODY CORPORATE, Denver, CO (US); ILLUMINA SOFTWARE, INC., SAN DIEGO, CA (US)

(72) Inventors: KATHLEEN BARNES, ENGLEWOOD, CO (US); IVANA YANG, ENGLEWOOD, CO (US); CHRISTOPHER GIGNOUX, CASTLE ROCK, CO (US); RASIKA MATHIAS, AURORA, CO (US); PAUL NORMAN, AURORA, CO (US); ALEM TAYE, SAN DIEGO, CA (US); RISHI PORECHA, SAN DIEGO, CA (US); BRET BARNES, SAN DIEGO, CA (US); BRETT PETERSON, BOULDER, CO (US)

(21) Appl. No.: 18/002,979

(22) PCT Filed: Jun. 23, 2021
(86) PCT No.: PCT/US2021/038763

§ 371 Date: Dec. 22, 2022

Related U.S. Application Data
(60) Provisional application No. 63/042,669, filed on Jun. 23, 2020.

(51) Int. Cl. C12Q 1/70; C12Q 1/6858

(52) U.S. Cl. CPC C12Q 1/701; C12Q 1/6858; C12Q 2600/118; C12Q 2600/154

(57) ABSTRACT

Provided by the inventive concept is a DNA methylation-based platform, and machine learning algorithms, for diagnosing respiratory pathogens including SARS-CoV-2 and predicting COVID-19 related outcomes, and methods of using the same, such as in identifying the presence of a viral infection, such as a SARS-CoV-2 infection, determining whether a subject has COVID-19, and/or whether a subject with COVID-19 is likely to develop acute respiratory distress syndrome or multisystem inflammatory syndrome in children.

### CROSS-REFERENCE TO RELATED APPLICATIONS

This application is a U.S.C. § 371 national phase application of PCT International Application No. PCT/US2021/038763, filed Jun. 23, 2021, which claims the benefit of U.S. Provisional Application Ser. No. 63/042,669, filed Jun. 23, 2020, the disclosures of each of which are incorporated herein by reference in their entireties.

### STATEMENT REGARDING ELECTRONIC FILING OF A SEQUENCE LISTING

This application includes a Sequence Listing which has been submitted electronically in ASCII text format. The ASCII copy, entitled 151077-00042US_ST25.txt, is 1,783,871 bytes in size, and was created on May 19, 2023, is provided in lieu of a paper copy. The Sequence Listing is incorporated herein by reference in its entirety into the specification for its disclosures.

### FIELD

The present inventive concept is related to a DNA methylation-based platform, methods to use the platform, and to diagnose and treat respiratory pathogens including SARS-CoV-2 and predict COVID-19 related complications and outcomes.

### SUMMARY

According to an aspect of the inventive concept, provided is a method of assaying for presence of a viral infection in a subject including: obtaining a methylation pattern for a set of DNA methylation sites from a sample derived from the subject; and analyzing the methylation pattern for the set of DNA methylation sites with an infection positive methylation classifier and/or an infection negative methylation classifier, wherein presence of a viral infection in the subject is indicated when a score derived from the infection positive classifier exceeds a cutoff and/or threshold value indicating the presence of a viral infection.

According to another aspect of the inventive concept, provided is a method for determining likelihood of a viral infection producing acute respiratory distress syndrome in a subject including: obtaining a methylation pattern for a set of DNA methylation sites from a sample derived from the subject; and analyzing the methylation pattern for the set of DNA methylation sites with an acute respiratory distress syndrome positive methylation classifier and/or an acute respiratory distress syndrome negative methylation classifier, wherein likelihood the subject will exhibit acute respiratory distress syndrome is indicated when a score derived from the acute respiratory distress syndrome positive classifier exceeds a cutoff and/or threshold value indicating likelihood of a viral infection producing acute respiratory distress syndrome.

According to another aspect of the invention, provided is a method of determining the nature of a viral infection in a subject including: obtaining a methylation pattern for a set of DNA methylation sites from a sample derived from the subject; and analyzing the methylation pattern for the set of DNA methylation sites with a SARS-CoV-2 positive methylation classifier and/or a SARS-CoV-2 negative methylation classifier, wherein presence of a SARS-CoV-2 infection in the subject is indicated when a score derived from the SARS-CoV-2 positive methylation classifier exceeds a cutoff and/or threshold value indicating presence of a SARS-CoV-2 infection.

According to another aspect of the inventive concept, provided is a method of treating COVID-19 in a subject including: obtaining a methylation pattern for a set of DNA methylation sites from a sample derived from the subject suspected of having COVID-19; analyzing the methylation pattern for the set of DNA methylation sites with a SARS-CoV-2 positive methylation classifier and/or a SARS-CoV-2 negative methylation classifier, wherein presence of a SARS-CoV-2 infection in the subject is indicated when a score derived from the SARS-CoV-2 positive methylation classifier exceeds a cutoff and/or threshold value indicating presence of a SARS-CoV-2 infection; and treating the subject for COVID-19 if the presence of a SARS-CoV-2 infection in the subject is indicated.

According to another aspect of the inventive concept, provided is a method of treating acute respiratory distress syndrome (ARDS) in a subject suspected of having COVID-19 including: obtaining a methylation pattern for a set of DNA methylation sites in a sample derived from the subject; analyzing the methylation pattern for the set of DNA methylation sites to an ARDS positive methylation classifier and/or an ARDS negative methylation classifier, wherein likelihood the subject will exhibit acute respiratory distress syndrome is indicated when a score derived from the ARDS positive classifier exceeds a cutoff and/or threshold value indicating likelihood of a COVID-19 infection producing ARDS; and treating the subject for ARDS.

According to another aspect of the inventive concept, provided is a method of treating multisystem inflammatory syndrome in adults (MIS-A) or multisystem inflammatory syndrome in children (MIS-C) in a subject suspected of having COVID-19 including: obtaining a methylation pattern for a set of DNA methylation sites in a sample derived from the subject; analyzing the methylation pattern for the set of DNA methylation sites to an MIS-A or an MIS-C positive methylation classifier, and/or an MIS-A or an MIS-C negative methylation classifier, wherein likelihood the subject will exhibit MIS-A or MIS-C is indicated when a score derived from the MIS-C positive classifier exceeds a cutoff and/or threshold value indicating likelihood of a COVID-19 infection producing MIS-A or MIS-C; and treating the subject for MIS-A or MIS-C.

Further aspects of the inventive concept include arrays, such as a methylation bead array, of DNA methylation sites, computer implemented methods for executing any of the methods presented hereinabove, and machine learning algorithms for performing the methods presented hereinabove.

### BRIEF DESCRIPTION OF THE DRAWINGS

FIG. 1. Overall callrates for COVID− (light gray) & COVID+ (dark gray) based on detection calibrated from neg. probe set on EPIC.

FIG. 2. Volcano plot depicting CpG sites significantly associated with COVID-19 status at FD-adjusted p-value<0.05 after adjustment for bias and inflation (dark gray and identified dots). Additional sites (light gray dots) were significant before adjustment for bias and inflation. Black dots were not significant.

FIG. 3. Application to COVID-19 or MIS-C for determination of a methylation-based epigenetic signature for COVID-19 or MIS-C.

FIG. 4. Approach to characterize epigenetic signatures using samples from children with COVID-19 or MIS-C.

FIG. 5. Training and testing of on COVID-19+ cases and COVID-19− controls to develop a classifier for the EPIC+ chip.

FIG. 6. SARS-CoV-2 disease symptomatic continuum that can be characterized according to methods of the inventive concept.

FIG. 7 is a block diagram of a classification system, computer program product, and/or computer-implemented method that may be used with a platform according to embodiments of the inventive concept.

FIG. 8 depicts an outline for classifier generation according to methods of the inventive concept.

FIG. 9. Approaches of machine learning used for classifier generation according to the inventive concept.

### DETAILED DESCRIPTION

Two stages of the inventive concept have been developed. The first includes a customized version of Illumina's Infinium Methylation EPIC BeadChip Kit (EPIC) for selecting 50,000 CpG sites/methylation probes that perform best at producing a COVID-19 diagnostic signature. To customize the EPIC chip, the CCPM team leveraged data generated from 25 nasopharyngeal swabs (NPS) of COVID-19+ and COVID-19− patients and added to those results CpG sites previously reported in the public domain and associated with respiratory viral infections and cardiopulmonary complications associated with recent coronavirus outbreaks, plus all 26,000 known HLA alleles, plus alternative haplotypes and unpublished reference sequences spanning the MHC genomic region, the Natural Killer Cell Immunoreceptor (KIR) and other immunogenetic loci, to enhance the sensitivity of immune response detection. Following manufacturing of the EPIC+ chip, quantitative methylation was performed on DNA samples from 624 patients testing positive or negative for COVID-19 using standard clinical practice (rtPCR testing). The second includes using ˜50K optimal CpG sites selected to create the Infinium HTS Custom Methylation COVID-19 Panel, that will reliably predict SARS-CoV-2 infection in whole blood by combining data from the methylation chip with a machine learning disease classifier that we have developed. In addition to accurately predicting SARS-CoV-2 infection in the host, the Infinium HTS Custom Methylation COVID-19 Panel, in combination with machine learning classifiers, will also (i) discriminate SARS-CoV-2 from other coronaviruses and respiratory viruses; (ii) predict which patients go on to develop clinical complications after primary infection (i.e., acute respiratory distress syndrome); and (iii) characterize signatures associated with both short- and long-term recovery. This invention addresses an unmet need of high-throughput and inexpensive tests for detecting the novel coronavirus causing COVID-19, at scale. Although high-throughput, rapid tests for detecting the novel coronavirus causing COVID-19 are being developed at an exceptional pace, rtPCR and serology tests are viral strain dependent, typical turnaround times for these assays range from 8-48 hours; a few tests have been developed for more rapid turnaround (30 minutes), yet they carry a high false negative rate (26-100%), their utility is limited to COVID-19, and there have been significant supply chain issues. Moreover, testing in general has been limited to symptomatic patients, a strategy that fails to address the critical need for screening and community wide surveillance. As such, there remains a need for more accurate and rapid tests for SARS-CoV-2 infection and for COVID-19 diagnosis.

Current standard of care for diagnosing SARS-CoV-2 is either by rtPCR upon infection or through serology (post infection). These methods are limited in that they will be viral strain dependent and require designing rtPCR primers to amplify and detect the SARS-CoV-2 virus. This is a binary detection procedure (positive or negative) for only SARS-CoV-2, that is unable to assess risk for an infected individual to develop a multitude of symptoms. The rtPCR tests are also limited by requiring validation using patient nasopharyngeal swabs. The platform of the present inventive concept improves on standard of care in the following areas: 1) detects SARS-CoV-2 viral infection by measuring genetic changes within the host using custom targeted Illumina epigenetic microarray chips, thus making it strain independent; 2) able to predict risk factors for a patient to be asymptomatic, mild symptom display, up to severe symptoms (e.g. acute respiratory distress syndrome (ADRS) and/or multisystem inflammatory syndrome in children (MIS-C)); 3) identify additional signatures to differentially diagnose for other respiratory viruses, including but not limited to: (i) respiratory syncytial virus (RSV); (ii) parainfluenza (1,2,3,4); (iii) human metapneumovirus (hMPV); (iv) human rhinovirus; (v) adenovirus (Ad); and (vi) extant coronaviruses (e.g., 229E (alpha coronavirus); NL63 (alpha coronavirus); OC43 (beta coronavirus); HKU1 (beta coronavirus), etc.); 4) host genomic samples are either able to be collected using upper airway (e.g., NPS) or peripheral blood. Primary areas of innovation are the testing strategy detecting viral infection using host epigenetic changes as the marker, potential capability to comprehensively identify other respiratory infections, and ability to predict patient symptomatic response to SARS-CoV-2.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a,” “an,” and “the” are used herein to refer to one or to more than one (i.e., at least one) of the grammatical object of the article. By way of example, “an element” means at least one element and can include more than one element. The term “and/or” includes any and all combinations of one, or more, of the associated listed items and may be abbreviated as “/”.

The term “comprise,” as used herein, in addition to its regular meaning, may also include, and, in some embodiments, may specifically refer to the expressions “consist essentially of” and/or “consist of” Thus, the expression “comprise” can also refer to embodiments, wherein that which is claimed “comprises” specifically listed elements does not include further elements, as well as embodiments wherein that which is claimed “comprises” specifically listed elements may and/or does encompass further elements, or encompass further elements that do not materially affect the basic and novel characteristic(s) of that which is claimed. For example, that which is claimed, such as a method, kit, system, etc. “comprising” specifically listed elements also encompasses, for example, a method, kit, system, etc. “consisting of,” i.e., wherein that which is claimed does not include further elements, and, for example, a method, kit, system, etc. “consisting essentially of,” i.e., wherein that which is claimed may include further elements that do not materially affect the basic and novel characteristic(s) of that which is claimed.

Unless otherwise defined, all technical terms used herein have the same meaning as commonly understood by one of ordinary skill in the art to which this disclosure belongs.

Methods of Testing, Diagnosing, Characterizing, and/or Predicting Outcomes

The present disclosure provides that alterations in genome-wide methylation patterns, in response to exposure respiratory viruses, such as SARS-CoV-2, can be used to identify and characterize, for example, presence of a viral infection, the nature of the viral infection, and/or probability for more severe manifestations of a viral infection in a subject with a high degree of accuracy.

The term “DNA methylation” and/or “gene methylation” refer to a biological process by which methyl groups are added to a DNA/nucleic acid molecule. DNA/gene/CpG island methylation may occur adenine (A) or cytosine (C), resulting in N⁶-methyladenosine (6 mA) for methylation of A, or 5-methylcytosine (5mC) and N⁴-methylcytosine (4mC) for C. In embodiments of the inventive concept, methylation levels of cytosine resulting in 5mC is measured/determined. In some embodiments, the extent of methylation of CpG dinucleotides in a DNA locus/gene is measured/determined. The methylated CpG dinucleotides may be located in CpG islands, regions in DNA that have a high frequency of CpG sites, as would be appreciated by one of skill in the art. In embodiments of the inventive concept, methylation of DNA/a set of gene loci and/or CpG islands located in DNA/gene/CpG island loci is measured in a subject and compared to DNA, gene, and/or CpG island methylation observed for populations having alternative biological states, e.g., a population having disease/infection vs. a population that is healthy/has no infection. In alternative biological states, e.g., disease/infection vs. healthy/no infection, in some embodiments, a particular DNA locus/gene may be hypomethylated, i.e., methylation of the DNA locus/gene in the disease/infection state is less than that of the healthy/no infection state, and in some embodiments, a particular DNA locus/gene may be hypermethylated, i.e., methylation of the DNA locus/gene in the disease/infection state is greater than that of the healthy/no infection state.

The term “signature,” according to embodiments of the inventive concept, can refer to a set of measurable quantities of biological markers, for example, genome-wide methylation patterns, genome-wide methylation of CpG islands, and/or methylation patterns of a set, e.g., a particular set and/or a pre-defined set, of genes/CpG islands, whose particular pattern/combination signifies the presence or absence of the specified biological state, such as a presence or absence of an infection or infections, such as, but not limited to: a SARS-CoV-2 infection, a respiratory syncytial virus (RSV) infection; a parainfluenza (1,2,3,4) infection; a human metapneumovirus (hMPV) infection; a human rhinovirus infection; an adenovirus (Ad) infection; and/or an extant coronavirus (e.g., 229E, alpha coronavirus; NL63, alpha coronavirus; OC43, beta coronavirus; HKU1, beta coronavirus) infection, or any combination thereof. In some embodiments, the particular pattern/combination signifies the presence or absence of a SARS-CoV-2 infection in a subject, and/or whether a subject is suffering from/afflicted with COVID-19, and/or the probability/likelihood the subject may be susceptible to and/is afflicted with more severe manifestations/symptoms of COVID-19, and/or a more severe condition related to the biological state, such as, acute respiratory distress syndrome (ARDS) and/or multiorgan failure in association with cytokine release and vascular leaks of immunopathology, e.g., multisystem inflammatory syndrome in adults (MIS-A) and/or multisystem inflammatory syndrome in children (MIS-C) associated with COVID-19. These signatures are discovered in a plurality of subjects with known status (e.g., COVID-19 positive, COVID-19 negative, confirmed with ARDS associated with COVID-19 and/or MIS-C associated with COVID-19), and are discriminative (individually or jointly) of one or more categories or outcomes of interest, for example, presence or absence of SARS-CoV-2, whether a subject is suffering from COVID-19, whether a subject is suffering from ARDS associated with COVID-19 and/or is more likely to suffer from ARDS associated with COVID-19, whether a subject is suffering from MIS-A associated with COVID-19 and/or is more likely to suffer from MIS-A associated with COVID-19, or whether a subject is suffering from MIS-C associated with COVID-19 and/or is more likely to suffer from MIS-C associated with COVID-19.

In some embodiments, a signature relates to a DNA/gene/CpG island methylation of a group/set of genes and/or methylation of CpG islands in the group/set of genes, for example, a set of genes including, but not necessarily limited to: ANLN, ARID3B, ARID5B, CALHM2, CBX3B2, CD38, CHSY1 CMPK2, DDX60, DTX3L, EPSTI1, FAM38A, FGFRL1, GPX1, GTPBP2, IFI27, IFIT3, IRF7, LINC00428, LINC01429, MX1, OAS1, OAS2, PARP9, PHOSPHO1, PPL, RAB40C, REPD, TNFRSF8, TRIM22, TSEN15, and ZDHHC6, or any subset thereof, whose methylation levels, when incorporated into a classifier as described herein, can discriminate the presence of specified biological states, such as, in some embodiments, presence or absence of SARS-CoV-2, whether a subject is suffering from COVID-19, whether a subject is suffering from ARDS associated with COVID-19 and/or is more likely to suffer from ARDS associated with COVID-19, or whether a subject is suffering from MIS-C associated with COVID-19 and/or is more likely to suffer from MIS-C associated with COVID-19. In some embodiments, a signature relates to a DNA/gene/CpG island methylation of a group/set of genes and/or methylation of CpG islands in the group/set of genes, for example, a set of genes including, but not necessarily limited to: ANLN, ARID3B, ARID5B, CALHM2, CBX3B2, CHSY1, DDX60, EPSTI1, FGFRL1, GPX1, IRF7, LINC00428, LINC01429, MX1, OAS1, OAS2, PARP9, PPL, RAB40C, REPD, TNFRSF8, TSEN15, and ZDHHC6, or any subset thereof. In some embodiments, the signature may include hypomethylation and/or hypermethylation of particular genes when comparing, for example, COVID-19+ and COVID-19− individuals. In some embodiments, the signature may include hypomethylation of IFR7, ARID5B, ANLN, PARP9, MX1, CBX3P2, EPSTI1, CHSY1, MX1, and/or GPX1, or any subset thereof. In some embodiments, the signature may include hypermethylation of LINC01429, CALHM2, LINC00428, OAS1, RAB40C, TSEN15, PEPD, PPL, ARID3B, ZDHHC6, TNFRSF8, DDX60, OAS2, and/or FGFRL1, or any subset thereof. In some embodiments, a signature relates to a DNA/gene/CpG island methylation of a group/set of genes and/or methylation of CpG islands in the group/set of genes, for example, a set of genes including, but not necessarily limited to: A_24_P561165; AA455656; AID; AIM2; ANLN; APE1; APOBEC3G; APOL2; APOL3; APOL6; ARID3B; ARID5B; ASK1; ATF2; B2M; BATF; BATF2; BCL2L14; C10orf81; C1R; C1S; C3; C4A; C4B; C5; C6; C7; C8; C9; CALHM2; CALR; CASP1; Caspase-3; Caspase-8; CBX3B2; CCRL1; CD19; CD4; CD74; CD8; CFH; CFHR1; CFHR3; CIITA; CIITA; CHSY1; BX117479; c-Jun; CLIC5; CTSS ZBP1; CX3CL1 A_24_P912985; CXCL10; CXCL11; CXCL2; DDX60; eIF-2; eIF2B; EPSTI1; ERP27; ETV7; FADD; FAM26F; FGFRL1; FZD5; GPX1; HCP5 NNMT; HLA-A; HLA-DMB; HLA-DOA; HLA-DPA1; HLA-DPB1; HLA-DQA1; HLA-DRA; HLA-DRB3; HLA-DRB4; HLA-DRB5; HLA-E; ICAM1; IFI16; IFI35; IFI44; IFI44L; IFIH1; IFIT2; IFIT3; IFIT5; IFITM1; IFNL1; IFNL2; IFNL3; IFNL4; IFN-α; IFN-β; IFNγ; IFN-γ; IFN-E; IFN-κ; IFN-ω; IGH; IGK; IGL; IKK-α; IKK-β; IKK-γ; IKKε; IL-10; IL-11; IL-12; IL12A; IL-13; IL-15; IL-17; IL-18; IL18BP; IL-1ra; IL-1α; IL-1β; IL-2; IL-3; IL-33; IL-36ra; IL-36α; IL-36β; IL-36γ; IL-37; IL-38; IL-4; IL-5; IL-6; IL-7; IL-8; IL-9; IRF1; IRF3; IRF7; JAK2; JNK; LAP3; LGP2; LINC00428; LINC01429; LT-α; M27126; MAVS; MDA5; MEKK1; MICA; MICAB; MKK7; MMP25; MX1; NAIP; NFKB1; NFKB2; NFKBIA; NLRC3; NLRC4; NLRC5; NLRP1; NLRP10; NLRP11; NLRP12; NLRP13; NLRP14; NLRP2; NLRP3; NLRP4; NLRP5; NLRP6; NLRP7; NLRP8; NLRP9; NLRX1; NMI; NOD1; NOD2; OAS1; OAS2; PARP9; PDIA3; PKR; PMAIP1; PML; POMC; PPL; PSMB8; PSMB9; RAB40C; REC8; REL; RELA; RELB; REPD; RIG-1; RIP; RNAse L; RTP4; SAMD9L; SECTM1; SEPX1; SERPING1; SOCS1; SP110; SPTLC3; SSTR2; STAT1; TAP1; TAP2; TAPBP; TBK1; TGFβ; TLR1; TLR10; TLR2; TLR3; TLR4; TLR5; TLR6; TLR7; TLR8; TLR9; TMEM140; TNF; TNFR1; TNFR2; TNFRSF8; TNFRSF14; TNFSF10; TNFSF13B; TP53; TRA; TRADD; TRAF2; TRAF3; TRB; TRD; TRG; TRIM25; TSEN15; UNG; VAMPS; WARS; XAF1; ZC3HAV1 and ZDHHC6, or any subset thereof. For example, in some embodiments, a subset of the group/set of genes, may include, for example: A_24_P561165; AA455656; AID; AIM2; APE1; APOBEC3G; APOL2; APOL3; APOL6; ASK1; ATF2; B2M; BATF; BATF2; BCL2L14; C10orf81; C1R; C1S; C3; C4A; C4B; C5; C6; C7; C8; C9; CALR; CASP1; Caspase-3; Caspase-8; CCRL1; CD19; CD4; CD74; CD8; CFH; CFHR1; CFHR3; CIITA; CIITA BX117479; c-Jun; CLIC5; CTSS ZBP1; CX3CL1 A _24_P912985; CXCL10; CXCL11; CXCL2; eIF-2; eIF2B; EPSTI1; ERP27; ETV7; FADD; FAM26F; FZD5; HCP5 NNMT; HLA-A; HLA-DMB; HLA-DOA; HLA-DPA1; HLA-DPB1; HLA-DQA1; HLA-DRA; HLA-DRB3; HLA-DRB4; HLA-DRB5; HLA-E; ICAM1; IFI16; IFI35; IFI44; IFI44L; IFIH1; IFIT2; IFIT3; IFIT5; IFITM1; IFNL1; IFNL2; IFNL3; IFNL4; IFN-α; IFN-β; IFNγ; IFN-γ; IFN-ε; IFN-κ; IFN-ω; IGH; IGK; IGL; IKK-α; IKK-β; IKK-γ; IKKε; IL-10; IL-11; IL-12; IL12A; IL-13; IL-15; IL-17; IL-18; IL18BP; IL-1ra; IL-1α; IL-1β; IL-2; IL-3; IL-33; IL-36ra; IL-36a; IL-3613; IL-36γ; IL-37; IL-38; IL-4; IL-5; IL-6; IL-7; IL-8; IL-9; IRF1; IRF3; IRF7; JAK2; JNK; LAP3; LGP2; LT-α; M27126; MAVS; MDA5; MEKK1; MICA; MICAB; MKK7; MMP25; MX1; NAIP; NFKB1; NFKB2; NFKBIA; NLRC3; NLRC4; NLRC5; NLRP1; NLRP10; NLRP11; NLRP12; NLRP13; NLRP14; NLRP2; NLRP3; NLRP4; NLRP5; NLRP6; NLRP7; NLRP8; NLRP9; NLRX1; NMI; NOD1; NOD2; OAS2; PDIA3; PKR; PMAIP1; PML; POMC; PSMB8; PSMB9; REC8; REL; RELA; RELB; RIG-1; RIP; RNAse L; RTP4; SAMD9L; SECTM1; SEPX1; SERPING1; SOCS1; SP110; SPTLC3; SSTR2; STAT1; TAP1; TAP2; TAPBP; TBK1; TGF13; TLR1; TLR10; TLR2; TLR3; TLR4; TLR5; TLR6; TLR7; TLR8; TLR9; TMEM140; TNF; TNFR1; TNFR2; TNFRSF14; TNFSF10; TNFSF13B; TP53; TRA; TRADD; TRAF2; TRAF3; TRB; TRD; TRG; TRIM25; UNG; VAMPS; WARS; XAF1; and ZC3HAV1, or any subset thereof.

As used herein, “array” can refer to a population of different microfeatures, such as microfeatures comprising polynucleotides, which are associated or attached with a surface such that the different microfeatures can be differentiated from each other according to relative location. An individual feature of an array can include a single copy of a microfeature or multiple copies of the microfeature can be present as a population of microfeatures at an individual feature of the array. The population of microfeatures at each feature typically is homogenous, having a single species of microfeature. Thus, multiple copies of a single nucleic acid sequence can be present at a feature, for example, on multiple nucleic acid molecules having the same sequence.

In some embodiments, a heterogeneous population of microfeatures can be present at a feature. Thus, a feature may but need not include only a single microfeature species and can instead contain a plurality of different microfeature species such as a mixture of nucleic acids having different sequences. Neighboring features of an array can be discrete one from the other in that they do not overlap. Accordingly, the features can be adjacent to each other or separated by a gap. In embodiments where features are spaced apart, neighboring sites can be separated, for example, by a distance of less than 100 pm, 50 pm, 10 pm, 5 pm, 1 pm, 0.5 pm, 100 nm, 50 nm, 10 nm, 5 nm, 1 nm, 0.5 nm, 100 pm, 50 pm, 1 pm or any distance within a range of any two of the foregoing distances. The layout of features on an array can also be understood in terms of center-to-center distances between neighboring features. An array useful in the invention can have neighboring features with center-to-center spacing of less than about 100 pm, 50 pm, 10 pm, 5 pm, 1 pm, 0.5 pm, 100 nm, 50 nm, 10 nm, 5 nm, 1 nm, 0.5 nm, 100 pm, 50 pm, 1 pm or any distance within a range of any two of the foregoing distances.

In some embodiments, the distance values described above and elsewhere herein can represent an average distance between neighboring features of an array. As such, not all neighboring features need to fall in the specified range unless specifically indicated to the contrary, for example, by a specific statement that the distance constitutes a threshold distance between all neighboring features of an array. Embodiments can be used with arrays having features at any of a variety of densities. Examples ranges of densities for certain embodiments include from about 10,000,000 features/cm² to about 2,000,000,000 features/cm²; from about 100,000,000 features/cm² to about 1,000,000,000 features/cm²; from about 100,000 features/cm² to about 10,000,000 features/cm²; from about 1,000,000 features/cm² to about 5,000,000 features/cm²; from about 10,000 features/cm² to about 100,000 features/cm²; from about 20,000 features/cm² to about 50,000 features/cm²; from about 1,000 features/cm² to about 5,000 features/cm², or any density within a range of any two of the foregoing densities.

As used herein, “surface” can refer to a part of a substrate or support structure that is accessible to contact with reagents, beads or analytes. The surface can be substantially flat or planar. Alternatively, the surface can be rounded or contoured. Example contours that can be included on a surface are wells, depressions, pillars, ridges, channels or the like. Example materials that can be used as a substrate or support structure include glass such as modified or functionalized glass; plastic such as acrylic, polystyrene or a copolymer of styrene and another material, polypropylene, polyethylene, polybutylene, polyurethane or TEFLON; polysaccharides or cross-linked polysaccharides such as agarose or Sepharose; nylon; nitrocellulose; resin; silica or silica-based materials including silicon and modified silicon; carbon-fiber; metal; inorganic glass; optical fiber bundle, or a variety of other polymers. A single material or mixture of several different materials can form a surface useful in the invention. In some embodiments, a surface comprises wells. In some embodiments, a support structure can include one or more layers. Example support structures can include a chip, a film, a multi-well plate, and a flow-cell.

As used herein, “bead” can refer to a small body made of a rigid or semi rigid material. The body can have a shape characterized, for example, as a sphere, oval, microsphere, or other recognized particle shape whether having regular or irregular dimensions. Example materials that are useful for beads include glass such as modified or functionalized glass; plastic such as acrylic, polystyrene or a copolymer of styrene and another material, polypropylene, polyethylene, polybutylene, polyurethane or TEFLON; polysaccharides or cross-linked polysaccharides such as agarose or Sepharose; nylon; nitrocellulose; resin; silica or silica-based materials including silicon and modified silicon; carbon-fiber; metal; inorganic glass; or a variety of other polymers. Example beads include controlled pore glass beads, paramagnetic beads, thoria sol, Sepharose beads, nanocrystals and others known in the art. Beads can be made of biological or non-biological materials. Magnetic beads are particularly useful due to the ease of manipulation of magnetic beads using magnets at various steps of the methods described herein. Beads used in certain embodiments can have a diameter, width, or length from about 0.1 pm to about 100 pm, from about 0.1 nm to about 500 nm. In some embodiments, beads used in certain embodiments can have a diameter, width or length less than about 100 pm, 50 pm, 10 pm, 5 pm, 1 pm, 0.5 pm, 100 nm, 50 nm, 10 nm, 5 nm, 1 nm, 0.5 nm, 100 pm, 50 pm, 1 pm or any diameter, width or length within a range of any two of the foregoing diameters, widths or lengths. Bead size can be selected to have reduced size, and hence get more features per unit area, whilst maintaining sufficient signal (template copies per feature) in order to analyze the features.

In some embodiments, polynucleotides can be attached to beads. In some embodiments, the beads can be distributed into wells on the surface of a substrate. Example bead arrays that can be used in certain embodiments include randomly ordered BEAD ARRAY technology (Illumina Inc., San Diego CA). Such bead arrays are disclosed in Michael et al, Anal Chem 70, 1242-8 (1998); Walt, Science 287, 451-2 (2000); Fan et al., Cold Spring Harb Symp Quant Biol 68:69-78 (2003); Gunderson et al., Nat Genet 37:549-54 (2005); Bibikova et al. Am J Pathol 165: 1799-807 (2004); Fan et al., Genome Res 14:878-85 (2004); Kuhn et al., Genome Res 14:2347-56 (2004); Yeakley et al., Nat Biotechnol 20:353-8 (2002); and Bibikova et al., Genome Res 16:383-93 (2006), each of which is incorporated by reference in its entirety.

As used herein, “target nucleic acid” or grammatical equivalent thereof can refer to nucleic acid molecules or sequences that it is desired to sequence, analyze and/or further manipulate. In some embodiments, a target nucleic acid can be attached to an array. In some embodiments, a capture probe can be attached to an array and the array used subsequently to detect a target nucleic acid in a sample that interacts with the probe. In this regard, it will be understood that in some embodiments, the terms “target” and “probe” can be used interchangeably with regard to nucleic acid detection methods.

In some embodiments, the number of different probes on an array range from 500 to 100,000. In other embodiments, the number of different targets on the array is at least 500, 1,000, 5,000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, or 1,000,000.

As used herein, “capture probe” can refer to a polynucleotide having sufficient complementarity to specifically hybridize to a target nucleic acid. A capture probe can function as an affinity binding molecule for isolation of a target nucleic acid from other nucleic acids and/or components in a mixture. In some embodiments, a target nucleic acid can be specifically bound by a capture probe through intervening molecules. Examples of intervening molecules include linkers, adapters and other bridging nucleic acids having sufficient complementarity to specifically hybridize to both a target sequence and a capture probe.

For the COVID-19 methylation analyses of the inventive concept, included are probes designed to target the following genes: ABCF1, ACBD5, AGL, AGPAT1, AIF1, ANKRD28, APOBEC3G, APOL6, APPBP2, ASPM, ATAT1, ATP2C1, B2M, BCL2L14, BRD2, C1orf68, C2, C4A, C4B, C6orf136, C6orf15, C7, CALR, CATSPER2, CATSPER2P1, CATSPERG, CCDC66, CCHCR1, CD27-AS1, CD4, CD40, CDKN1A, CELF4, CEP162, CFB, CFH, CFHR1, CFHR2, CFHR3, CFHR4, CHTOP, CIITA, CSNK2B, CTNND1, CUTA, CYP21A2, DAG1, DDR1, DENND2B, DEXI, DTNB, DYSF, E2F5, EGFL8, EHMT2, ELAVL2, FAM49B, FKBP5, FLG, FLG-AS1, FRMD3, GAPVD1, GPANK1, GPX5, GTF2H4, HCG17, HCG18, HCG20, HCG22, HCG24, HCG25, HCG27, HCG4, HCG4B, HCP5, HLA-A, HLA-B, HLA-C, HLA-DPA1, HLA-DPB1, HLA-DPB2, HLA-DQA1, HLA-DQA2, HLA-DQB1, HLA-DQB1-AS1, HLA-DRA, HLA-E, HLA-F, HLA-F-AS1, HLA-G, HLA-H, HLA-V, HRNR, HSD17B8, HSPA1B, ICAM1, IFI16, IFI35, IFI44, IFI44L, IFITM1, IVL, JAK2, KCTD16, KDM4C, KIFC1, KPRP, LAP3, LCE1D, LCE1E, LCE2B, LCE2C, LCE2D, LCE3C, LCE4A, LELP1, LINC00302, LINC01185, LOC100129636, LOC100294145, LOC100507547, LOC101929006, LOC105375690, LRBA, LST1, LTA, LTBR, LY6G6F-LY6G6D, LYRM4-AS1, MCCD1, MDC1, MEF2C, MICA, MICA-AS1, MICB, MICB-DT, MIR219A1, MIR4479, MMP25, MOG, MPIG6B, MSH5, MUCL3, MX1, NA, NBPF18P, NELFE, NFKB1, NFKB2, NLRC5, NLRP11, NLRP2, NLRP3, NLRP4, NLRP5, NLRX1, NRCAM, OR10C1, OR12D2, OR14J1, OR2H1, OR2H2, OR2J1, OR2J2, OSMR, PCDH15, PDCD6IPP2, PDIA3, PGLYRP4, PHF1, PML, PPP1R11, PPP2R5A, PPP6R3, PPT2, PRR3, PRR9, PRRC2A, PRRT1, PSMB8-AS1, PSMB9, PSORS1C1, PSORS1C2, RALYL, REC8, REL, RELB, RING1, RNF43, RNF5, RNF5P1, RPL13A, RP S18, RPS6KC1, RTP4, RXRB, S100A13, S100A7, S100A8, SCNN1A, SERPINB7, SERPING1, SHMT1, SKIV2L, SLC44A4, SLCO5A1, SMURF2P1-LRRC37BP1, SNAPC3, SNHG32, SNORD32B, SNX14, SPRR1A, SPRR1B, SPRR3, SPRR4, SPTLC3, STAT4, STOML1, SUN1, SYNGAP1, TAP1, TBC1D5, TBK1, TCF19, THRB, TK2, TLR2, TLR3, TLR4, TMEM185A, TMEM62, TNF, TNFRSF14, TNFRSF14-AS1, TNFSF13B, TPTE2P5, TRAF2, TRAF3, TRIM10, TRIM15, TRIM27, TRIM33, TRIM37, TRIM39, TRIM40, TRIT1, TRPM4, TSBP1, TSBP1-AS1, TUBB, UBE2K, USP8, VARS2, VEZT, VPS52, XAF1, XPOT, YY1AP1, ZMYND11, ZNF248, ZNF512, ZNF610, and ZNRD1ASP. Sequences for probes to these genes include those as set forth in SEQ ID NOS:1-7,831 of the accompanying Sequence Listing, entitled IP-2201-PCT_SL.txt, or any subset thereof.

In some embodiments, the probes include at least one sequence from the group consisting of SEQ ID NOS:42, 48, 49, 56, 60, 152, 153, 154, 155, 156, 160, 161, 170, 174, 175, 176, 192, 195, 196, 205, 206, 207, 208, 209, 210, 211, 217, 219, 220, 221, 222, 235, 255, 294, 295, 298, 299, 300, 310, 315, 322, 329, 330, 331, 337, 602, 607, 608, 668, 669, 677, 678, 738, 750, 751, 756, 757, 761, 762, 769, 770, 773, 776, 777, 779, 829, 830, 842, 843, 846, 847, 855, 856, 857, 858, 860, 864, 869, 870, 877, 882, 904, 905, 916, 922, 923, 924, 925, 933, 942, 943, 959, 964, 965, 966, 969, 981, 982, 999, 1000, 1002, 1003, 1004, 1005, 1035, 1036, 1046, 1047, 1048, 1049, 1062, 1063, 1090, 1095, 1096, 1097, 1122, 1123, 1138, 1145, 1146, 1155, 1156, 1158, 1165, 1173, 1174, 1180, 1181, 1185, 1210, 1211, 1216, 1217, 1219, 1220, 1225, 1237, 1238, 1247, 1248, 1249, 1250, 1254, 1255, 1256, 1259, 1260, 1263, 1270, 1271, 1296, 1297, 1308, 1309, 1312, 1314, 1318, 1319, 1326, 1328, 1329, 1342, 1348, 1405, 1406, 1413, 1414, 1415, 1416, 1433, 1434, 1443, 1444, 1465, 1470, 1476, 1485, 1486, 1487, 1508, 1509, 1510, 1514, 1612, 1613, 1620, 1627, 1638, 1639, 1664, 1665, 1666, 1669, 1670, 1676, 1677, 1684, 1685, 1701, 1712, 1719, 1720, 1721, 1722, 1726, 1727, 1731, 1738, 1740, 1747, 1748, 1852, 1853, 1854, 1939, 2072, 2073, 2075, 2088, 2090, 2171, 2193, 2194, 2434, 2435, 2604, 2676, 2678, 2680, 2681, 2756, 2914, 2915, 2919, 2920, 3180, 3243, 3774, 3775, 3994, 3995, 3996, 3998, 3999, 4000, 4020, 4021, 4030, 4031, 4046, 4103, 4171, 4179, 4184, 4187, 4188, 4225, 4236, 4251, 4253, 4257, 4258, 4259, 4262, 4263, 4272, 4276, 4277, 4278, 4292, 4293, 4313, 4314, 4316, 4317, 4318, 4328, 4329, 4330, 4343, 4344, 4345, 4357, 4358, 4376, 4377, 4384, 4389, 4390, 4402, 4408, 4409, 4410, 4411, 4414, 4415, 4416, 4418, 4419, 4421, 4422, 4426, 4427, 4430, 4439, 4453, 4454, 4456, 4457, 4458, 4479, 4487, 4488, 4491, 4492, 4493, 4494, 4500, 4501, 4518, 4519, 4525, 4526, 4539, 4540, 4555, 4562, 4563, 4564, 4584, 4585, 4586, 4589, 4594, 4595, 4596, 4597, 4615, 4617, 4618, 4619, 4620, 4621, 4622, 4627, 4628, 4629, 4630, 4631, 4632, 4657, 4658, 4661, 4662, 4671, 4673, 4689, 4690, 4691, 4697, 4698, 4716, 4717, 4726, 4727, 4728, 4729, 4731, 4747, 4768, 4773, 4774, 4778, 4779, 4780, 4781, 4782, 4783, 4784, 4785, 4968, 4969, 4976, 4977, 4987, 4993, 4994, 4995, 4999, 5005, 5006, 5020, 5025, 5026, 5027, 5035, 5049, 5050, 5055, 5056, 5158, 5164, 5171, 5172, 5173, 5188, 5189, 5190, 5191, 5192, 5193, 5204, 5206, 5208, 5209, 5210, 5211, 5212, 5213, 5214, 5217, 5219, 5220, 5225, 5232, 5233, 5234, 5235, 5238, 5239, 5240, 5241, 5294, 5295, 5296, 5313, 5316, 5327, 5370, 5375, 5376, 5377, 5378, 5379, 5385, 5507, 5508, 5509, 5510, 5511, 5512, 5513, 5514, 5515, 5516, 5517, 5561, 5572, 5573, 5574, 5577, 5578, 5579, 5585, 5586, 5592, 5645, 5646, 5649, 5650, 5656, 5657, 5667, 5672, 5681, 5684, 5695, 5696, 5697, 5698, 5699, 5700, 5701, 5702, 5710, 5711, 5720, 5725, 5728, 5729, 5730, 5743, 5744, 5745, 5748, 5749, 5759, 5760, 5761, 5765, 5766, 5768, 5769, 5772, 5780, 5781, 5782, 5802, 5803, 5804, 5807, 5808, 5809, 5813, 5814, 5816, 5817, 5828, 5829, 5833, 5910, 5914, 5917, 5937, 5941, 5942, 5944, 5945, 5948, 5949, 5963, 6018, 6019, 6023, 6031, 6032, 6033, 6039, 6040, 6043, 6045, 6109, 6110, 6111, 6112, 6113, 6116, 6127, 6128, 6133, 6134, 6137, 6225, 6236, 6242, 6449, 6450, 6451, 6452, 6453, 6454, 6455, 6457, 6458, 6461, 6462, 6463, 6466, 6469, 6470, 6471, 6480, 6481, 6545, 6546, 6547, 6613, 6684, 6685, 6692, 6693, 6694, 6695, 6710, 6711, 6731, 6732, 6741, 6787, 6788, 6805, 6806, 6807, 6828, 6829, 6830, 6831, 6832, 6835, 6836, 6846, 6847, 6848, 6849, 6850, 6862, 6868, 6869, 6870, 6871, 6878, 6879, 6897, 6898, 6899, 6900, 6908, 6909, 6914, 6938, 6939, 6949, 6950, 6951, 6952, 6959, 6960, 6971, 6972, 6973, 6974, 6976, 6979, 7110, 7111, 7112, 7113, 7117, 7118, 7120, 7122, 7124, 7135, 7184, 7185, 7401, 7402, 7404, 7408, 7441, 7442, 7482, 7490, 7491, 7497, 7500, 7503, 7504, 7513, 7514, 7515, 7525, 7526, 7621, 7622, 7623, 7624, 7625, 7626, 7627, 7638, 7649, 7650, 7651, 7652, 7665, 7694, 7704, 7705, 7708, 7716, 7717, 7718, 7726, 7727, 7728, 7729, 7738, 7739, 7740, 7741, 7742, 7743, 7744, 7746, 7747, 7749, 7757, 7770, 7771, 7774, 7775, 7777, 7783, 7788, 7789, 7790, 7791, 7798, 7799, 7803, 7804, 7815, 7816, 7823, 7824, and 7825 of the accompanying Sequence Listing, or any subset thereof.

In some embodiments, the methylation signature includes detecting methylation status of at least one gene selected from the group consisting of: ABCF1, ABCF1, AIF1, APOBEC3G, APOL6, B2M, BCL2L14, BRD2, C2, C6orf136, C6orf15, C7, CALR, CD27-AS1, CD4, CD40, CFB, CFH, CHTOP, CIITA, CSNK2B, CUTA, CYP21A2, DDR1, DEXI, EGFL8, EHMT2, GPANK1, GPX5, GTF2H4, HCG17, HCG18, HCG20, HCG25, HCG27, HCG4, HCG4B, HLA-A, HLA-DPA1, HLA-DPB2, HLA-DQA2, HLA-E, HLA-F, HLA-F-AS1, HLA-G, HLA-V, ICAM1, IFI16, IFI35, IFI44, IFI44L, IFITM1, IVL, KIFC1, KPRP, LCE1D, LCE1E, LCE2C, LELP1, LINC00302, LOC100507547, LOC101929006, LY6G6F-LY6G6D, MICA-AS1, MICB, MICB-DT, MSH5, MX1, NA, NBPF18P, NFKB1, NFKB2, NLRC5, NLRP11, NLRP3, NLRP5, NLRX1, OR2H1, OSMR, PDIA3, PHF1, PML, PPP1R11, PPT2, PRR9, PRRC2A, PSMB8-AS1, PSMB9, PSORS1C1, REC8, REL, RELB, RING1, RNF5, RPS18, RTP4, RXRB, S100A13, SCNN1A, SERPING1, SKIV2L, SLC44A4, SNORD32B, SPRR4, SPTLC3, SYNGAP1, TAP1, TBK1, TCF19, TLR3, TNF, TNFRSF14, TNFSF13B, TRAF2, TRAF3, TRIM15, TRIM27, TRIM39, TSBP1-AS1, TUBB, VARS2, VPS52, XAF1, and ZNRD1ASP, or any subset thereof.

The terms “classifier” and “predictor” may be used interchangeably and refer to a process for determining a category that a sample may be assigned to, that uses the values of the signature (e.g., methylation levels for a set of genes) and a pre-determined coefficient (or weight) for each signature component to generate scores for a given observation or individual patient for the purpose of assignment to a category. The classifier may be linear and/or probabilistic. A classifier is linear if scores are a function of summed signature values weighted by a set of coefficients. Furthermore, a classifier is probabilistic if the function of signature values generates a probability, a value between 0 and 1.0 (or 0 and 100%) quantifying the likelihood that a subject or observation belongs to a particular category or will have a particular outcome, respectively. Probit regression and logistic regression are examples of probabilistic linear classifiers that use probit and logistic link functions, respectively, to generate a probability. In some embodiments, the classifier may be a process to bin samples into categories depending on specified measured characteristics, for example, observed DNA/gene/CpG island methylation levels.

The classifier equation may take, for example, the general form:

P(having condition)=Φ(β₁X₁+β₂X₂+ . . . +β_{d}X_{d})

wherein the condition is, e.g., presence of a SARS-CoV-2 infection, and/or susceptibility for ARDS/MIS-A/MIS-C. Φ(.) is the probit (or logistic, etc.) link function; {β₁, β₂, . . . , β_{d}} are the coefficients obtained through training of the classifier when the host response biomarker is translated to the platform (the coefficients may also be denoted {w₁, w₂, . . . , w_{d}} as “weights” herein); {X₁, X₂, . . . , X_{d}} are the DNA/gene/CpG island methylation levels of the signature/biomarker; and d is the size of the signature/biomarker (i.e., number of methylation sites/loci).

It should be noted that the threshold or cutoff value may be adjusted to accommodate the diagnostic decision. For example, the threshold for diagnosing a bacterial infection may be lowered to favor test sensitivity and thus reduce the possibility of a potentially life-threatening false negative result.

A classifier may be developed by a procedure known as “training,” which makes use of a set of data containing observations, for example, DNA/gene/CpG island methylation levels, with known category membership. Specifically, training seeks to find the optimal coefficient (i.e., weight) for each component of a given signature (e.g., DNA/gene/CpG island methylation levels and differential DNA/gene/CpG island methylation levels of components), as well as an optimal signature, such as a set of genes/biomarkers, where the optimal result is determined by the highest achievable classification accuracy. DNA/CpG islands with higher degrees of methylation differences between positive cases and negative controls are typically the probes that are selected as features/components for the signature. Classifiers of the inventive concept may be generated, for example, by iteratively: assigning a weight for the extent of methylation of each DNA locus/gene, entering the weight and value for the extent of methylation of each DNA locus/gene into a classifier equation and determining a score for outcome for each of a plurality of subjects; determining the accuracy of classification for each outcome across the plurality of subjects, and adjusting the weight until accuracy of classification is optimized, to provide the classifier. Classifiers of the inventive concept include, for example, classifiers for whether a subject patient is infected with SARS-CoV-2, whether a subject is suffering from COVID-19, whether a subject is suffering from ARDS associated with COVID-19 and/or is more likely to suffer from ARDS associated with COVID-19, or whether a subject is suffering from MIS-C associated with COVID-19 and/or is more likely to suffer from MIS-C associated with COVID-19. The classifiers that are developed during training, using a training set of samples, are applied for prediction purposes to diagnose new individuals (“classification”).

In some embodiments, classifiers of the inventive concept may be developed/generated using a support-vector machine/machines (SVM/SVMs). Any SVM available may be used for generating the classifier as would be appreciated by one of skill in the art. Software, for example, svmlib/libsvm may be used for training and/or optimization of the classifier. Improving performance of the classifier may be accomplished by either better feature selection (DNA/gene/CpG island methylation sites/components of the signature), such as selecting DNA/CpG islands with higher degrees of methylation differences between positive cases and negative controls, or by gathering further data/observations.

“Classification” may refer to a method of assigning a subject suffering from or at risk for symptoms to one or more categories or outcomes (e.g., a whether subject/patient is infected with SARS-CoV-2, whether a subject is suffering from COVID-19, whether a subject is suffering from ARDS associated with COVID-19 and/or is more likely to suffer from ARDS associated with COVID-19, whether a subject is suffering from MIS-A associated with COVID-19 and/or is more likely to suffer from MIS-A associated with COVID-19, or whether a subject is suffering from MIS-C associated with COVID-19 and/or is more likely to suffer from MIS-C associated with COVID-19). In some cases, a subject may be classified to more than one category, e.g., in case of suffering from COVID-19 and is more likely to suffer from MIS-C associated with COVID-19. The outcome, or category, is determined by the value of the scores provided by/derived from the classifier, which may be compared to a cutoff or threshold value, confidence level, or limit. In other scenarios, the probability of belonging to a particular category may be given (e.g., if the classifier reports probabilities). In some embodiments, a high probability or likelihood reported by the classifier may be about 0.7 or greater, may be about 0.75 or greater, about 0.8 or greater, about 0.85 or greater, about 0.9 or greater, about 0.95 or greater, about 0.98 or greater, or about 0.99 or greater. In some embodiments a high percentage likelihood reported by the classifier may be about 70% or greater, about 75% or greater, about 80% or greater, about 85% or greater, about 90% or greater, about 95% or greater, about 98% or greater, or about 99% or greater.

The term “indicative,” when used with DNA/gene/CpG island methylation levels, can mean that the DNA/gene/CpG island methylation levels are up-regulated or down-regulated, altered, or changed compared to the levels in alternative biological states (e.g., whether or not a patient/subject is infected with SARS-CoV-2, whether a subject is suffering from COVID-19, whether a subject is suffering from ARDS associated with COVID-19 and/or is more likely to suffer from ARDS associated with COVID-19, or whether a subject is suffering from MIS-C associated with COVID-19 and/or is more likely to suffer from MIS-C associated with COVID-19) or control. The term “indicative” when used with DNA/gene/CpG island methylation levels means that the DNA/gene/CpG island methylation levels are higher or lower, increased or decreased, altered, or changed compared to the standard protein levels or levels in alternative biological states. Measured DNA/gene/CpG island methylation levels, when analyzed with pre-determining weights in the context of a classifier, such as a classifier for a presence of SARS-CoV-2, whether a subject is suffering from COVID-19, i.e., disease associated with SARS-CoV-2, whether a subject is suffering from more severe symptoms associated with COVID-19 and/or is more likely to suffer from more severe symptoms associated with COVID-19, whether a subject is suffering from ARDS associated with COVID-19 and/or is more likely to suffer from ARDS associated with COVID-19, or whether a subject is suffering from MIS-C associated with COVID-19 and/or is more likely to suffer from MIS-C associated with COVID-19 as described herein, may provide a score/outcome/result “indicative” of the presence of SARS-CoV-2, whether a subject is suffering from COVID-19, whether a subject is suffering from ARDS associated with COVID-19 and/or is more likely to suffer from ARDS associated with COVID-19, or whether a subject is suffering from MIS-C associated with COVID-19 and/or is more likely to suffer from MIS-C associated with COVID-19.

It will be appreciated that symptoms for SARS-CoV-2 disease (COVID-19) spread across a spectrum/continuum of states, including asymptomatic disease. Symptoms may include, but are not limited to, for example: fever and/or chills; cough; shortness of breath and/or difficulty breathing; fatigue; muscle and/or body aches; headache; new loss of taste and/or smell; sore throat; congestion and/or runny nose; nausea and/or vomiting; and diarrhea. More severe symptoms may include, but are not limited to, symptoms that may require immediate emergency medical care, for example: trouble breathing; persistent pain or pressure in the chest; new confusion; inability to wake or stay awake; and, depending on skin tone, pale, gray, or blue-colored skin, lips, or nail beds. Severe SARS-CoV-2 disease may also include ARDS, MIS-A, and/or MIS-C associated with COVID-19.

The terms “subject” and “patient” may be used interchangeably and refer to any animal being examined, studied, or treated. It is not intended that the present disclosure be limited to any particular type of subject. In some embodiments of the present invention, humans are the preferred subject, while in other embodiments non-human animals are the preferred subject, including, but not limited to, mice, monkeys, ferrets, cattle, sheep, goats, pigs, chicken, turkeys, dogs, cats, horses and reptiles, and for example, a laboratory animal such as a rat, mouse, guinea pig, rabbit, primates, etc.), a farm or commercial animal (e.g., a cow, pig, horse, goat, donkey, sheep, etc.), or a domestic animal (e.g., cat, dog, ferret, horse, etc.). Human subjects may be of any gender (for example, male, female or transgender) and at any stage of development (i.e., neonate, infant, juvenile, adolescent, adult, elderly). In some embodiments, the subject may be a human subject that may be suffering from more severe symptoms associated with COVID-19 and/or is more likely to suffer from more severe symptoms associated with COVID-19. In some embodiments, the subject may be a human subject that may be suffering from ARDS associated with COVID-19 and/or is more likely to suffer from ARDS associated with COVID-19. In some embodiments, the subject may be an adult or elderly human subject that may be suffering from MIS-A associated with COVID-19 and/or is more likely to suffer from MIS-A associated with COVID-19. In some embodiments, the subject may be a non-adult or non-elderly human subject (i.e., a neonate, infant, juvenile, or adolescent human subject) that may be suffering from MIS-C associated with COVID-19 and/or is more likely to suffer from MIS-C associated with COVID-19.

In some embodiments, the subject is at high risk for contracting a coronavirus, such as SARS-CoV-2, and/or for suffering from more severe symptoms associated with SARS-CoV-2 disease. In some embodiments, the subject is aged 65 or older, has high blood pressure, asthma, lung disease, cancer, diabetes, Down syndrome, heart disease/conditions, HIV, kidney disease, liver disease, lung disease, sickle cell disease or thalassemia, a neurological condition such as dementia, a substance use disorder, had a solid organ or blood stem cell transplant, and/or had a stroke/cerebrovascular disease, is pregnant, is overweight/obese, smokes, and/or is immunocompromised. In some embodiments, the immunocompromised subject may have an immunodeficiency disease and/or may have a deficiency in Type I IFN defenses.

A “platform” or “technology” refers to an apparatus (e.g., instrument and associated parts, computer, computer-readable media including one or more databases as taught herein, reagents, arrays, etc.) that may be used to measure a signature, e.g., DNA/gene/CpG island methylation levels, in accordance with the inventive concept. Examples of platforms for analyzing/measuring DNA/gene/CpG island methylation levels may include methylation bead chips. Exemplary methylation bead chips include for example, commercial platforms, such as the Illumina Infinium Methylation EPIC BeadChip Kit, and custom platforms, such as a customized Illumina Infinium Methylation EPIC BeadChip Kit (EPIC+), and an Illumina Infinium HTS Custom Methylation COVID-19 Panel as described herein.

In some embodiments, the platform may be configured to measure DNA/gene/CpG island methylation levels semi-quantitatively, i.e., rather than measuring discrete or absolute DNA/gene/CpG island methylation levels, the DNA/gene/CpG island methylation levels are measured as an estimate and/or relative to each other or a specified marker or markers (e.g., DNA/gene/CpG island methylation of another, “standard” or “reference” gene/marker).

Analysis of DNA/gene/CpG island methylation, according to embodiments of the inventive concept, may include treating DNA with bisulfite, e.g., sodium bisulfite prior to nucleic acid amplification/methylation analysis. Analysis of DNA/gene/CpG island methylation, according to embodiments of the inventive concept, may include nucleic acid amplification of bisulfite-treated DNA. Nucleic acid amplification, according to embodiments of the inventive concept, may be accomplished by any method that would be appreciated by one of skill in the art. In some embodiments, nucleic acid amplification may include whole genome amplification (WGA) of bisulfite-treated DNA by way of, for example, random hexamer primer priming and and Phi29 polymerase and enzymatic fragmentation of amplification products prior to DNA/gene/CpG island methylation analysis. Nucleic acid amplification products of bisulfite-treated DNA may then be analyzed with a platform or technology as described herein.

Nucleic acid amplification may include thermal amplification, such as Polymerase Chain Reaction (PCR), or may be include isothermal amplification, such as Loop-Mediated Isothermal Amplification (LAMP), Multiple Displacement Amplification (MDA), Strand Displacement Amplification (SDA), Helicase-Dependent Amplification (HDA), Recombinase Polymerase Amplification (RPA), Nucleic Acid Sequences Based Amplification (NASBA), Rolling Circle Amplification (RCA).

The term “biological sample” includes any sample that may be taken from a subject/biological source that contains genetic material that can be used in the methods provided herein. For example, a biological sample may include a blood sample, such as a peripheral blood sample. The term “peripheral blood sample” refers to a sample of blood circulating in the circulatory system or body taken from the system of body. Other samples may include those taken from the upper respiratory tract, including but not limited to, sputum, nasopharyngeal swab (NPS) and nasopharyngeal wash, or synovial fluid, or cerebrospinal fluid. A biological sample may also include those samples taken from the lower respiratory tract, including but not limited to, sputum, bronchoalveolar lavage and endotracheal aspirate. A biological sample may also include any combinations thereof. A “biological source” includes, for example, human or non-human subjects (“in vivo”), cultured cells (“in vitro”), and primary human tissues (“ex vivo”) from which a sample/biological sample may be obtained/derived from. Measurements/determinations/analysis of, for example, DNA/gene/CpG island methylation levels of genes, in a biological source or in biological sources include, and may be provided by, in some embodiments, measurements/determinations/analysis of DNA/gene/CpG island methylation levels of genes in a sample/biological sample derived from the biological source.

The terms “obtaining,” “gathering,” and/or “collecting,” when referring to methylation levels of genes and/or DNA methylation levels may include experimentally measuring methylation levels of DNA/gene/CpG island methylation levels in, for example, a sample/biological sample derived from, for example, a biological source, as well as drawing measured/determined DNA/gene/CpG island methylation levels from, for example, public and/or commercially available databases of DNA/gene/CpG island methylation data that are or will be available to one of skill in the art. The terms “obtaining,” “gathering,” and/or “collecting,” when referring to a sample, such as a biological sample, may include experimentally obtained, gathered, and/or collected samples from a source, such as a biological source, as well samples drawn from, for example, publicly available and/or commercial repositories as will be appreciated by one of skill in the art.

The terms “treat”, “treatment” and “treating” refer to the reduction or amelioration of the severity, duration and/or progression of a disease or disorder, or one or more symptoms thereof resulting from the administration of one or more therapies. Such terms may refer to a reduction in the replication of pathogens, such as respiratory viruses as described herein, or a reduction in the spread of pathogens to other organs or tissues in a subject or to other subjects.

An “appropriate treatment regimen” refers to the standard of care needed to treat a specific disease or disorder. Often such regimens require the act of administering to a subject a therapeutic agent(s) capable of producing a curative effect in a disease state. For example, an appropriate treatment regimen may include administration of any therapeutic agent for treatment of pathogens, for example, respiratory viruses as described herein, such as, but not limited to: SARS-CoV-2 (the coronavirus associated with coronavirus disease 2019, or COVID-19); respiratory syncytial virus (RSV); parainfluenza (1,2,3,4); human metapneumovirus (hMPV); human rhinovirus; adenovirus (Ad); and extant coronaviruses, e.g., 229E (alpha coronavirus); NL63 (alpha coronavirus); OC43 (beta coronavirus); HKU1 (beta coronavirus); MERS-CoV (the beta coronavirus associated with Middle East Respiratory Syndrome, or MERS); and/or SARS-CoV (the beta coronavirus associated with severe acute respiratory syndrome, or SARS), in an appropriate amount as would be appreciated by one of skill in the art. The inventive concept further contemplates the use of methods according to the inventive concept to determine treatments of such pathogens with therapeutics, that are not yet available. In some embodiments, the inventive concept contemplates treating and/or preventing any virus/viral infection belonging to the Coronaviridae family now known or yet to be discovered.

### Systems and Computer-Implemented Methods

A classification system, computer program product, and/or computer-implemented methods may be used in or by a platform, according to various embodiments described herein. A classification system, computer program product, and/or computer-implemented method may be embodied as one or more enterprise, application, personal, pervasive and/or embedded computer systems that are operable to receive, transmit, process and store data using any suitable combination of software, firmware and/or hardware and that may be standalone and/or interconnected by any conventional, public and/or private, real and/or virtual, wired and/or wireless network including all or a portion of the global communication network known as the Internet, and may include various types of tangible, non-transitory computer readable medium. Hardware on which classification systems, computer program products and/or computer-implemented methods of the inventive concept may be used is not particularly limited, and may include, without limitation, personal computers, handheld and/or mobile devices, phones, etc. In some embodiments, the systems, computer programs, and/or compute-implemented methods of the inventive concept may be cloud-based.

The classification system may include a processor subsystem, including one or more Central Processing Units (CPU) on which one or more operating systems and/or one or more applications run. It will be understood that multiple processors may be present, which may be either electrically interconnected or separate. Processor(s) are configured to execute computer program code from memory devices, such as memory, to perform at least some of the operations and methods described herein, and may be any conventional or special purpose processor, including, but not limited to, digital signal processor (DSP), field programmable gate array (FPGA), application specific integrated circuit (ASIC), and multi-core processors.

The memory subsystem may include a hierarchy of memory devices such as random-access memory (RAM), read-only memory (ROM), erasable programmable read-only memory (EPROM) or flash memory, and/or any other solid state memory devices.

A storage circuit may also be provided, which may include, for example, a portable computer diskette, a hard disk, a portable compact disk read-only memory (CDROM), an optical storage device, a magnetic storage device and/or any other kind of disk- or tape-based storage subsystem. The storage circuit may be provided on hardware including, but not limited to, computers, such as personal computers (PCs), mobile/handheld devices, such as tablets and/or mobile phones, etc., or may be provided on the cloud. The storage circuit may provide non-volatile storage of data/parameters/classifiers for the classification system. The storage circuit may include disk drive and/or network store components. The storage circuit may be used to store code to be executed and/or data to be accessed by the processor. In some embodiments, the storage circuit may store databases which provide access to the data/parameters/classifiers used for the classification system such as the signatures, weights, thresholds, etc. Any combination of one or more computer readable media may be utilized by the storage circuit. The computer readable media may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: a portable computer diskette, a hard disk, a random-access memory (RAM), a read-only memory (ROM), an erasable programmable read-only memory (EPROM or flash memory), a portable compact disc read-only memory (CD-ROM), an optical storage device, a magnetic storage device, or any suitable combination of the foregoing. As used herein, a computer readable storage medium may be any tangible medium that can contain or store a program for use by or in connection with an instruction execution system, apparatus, or device.

An input/output circuit may include displays and/or user input devices, such as keyboards, touch screens and/or pointing devices. Devices attached to the input/output circuit may be used to provide information to the processor by a user of the classification system. Devices attached to the input/output circuit may include networking or communication controllers, input devices (keyboard, a mouse, touch screen, etc.) and output devices (printer or display). The input/output circuit may also provide an interface to devices, such as a display and/or printer, to which results of the operations of the classification system can be communicated so as to be provided to the user of the classification system.

An optional update circuit may be included as an interface for providing updates to the classification system. Updates may include updates to the code executed by the processor that are stored in the memory and/or the storage circuit. Updates provided via the update circuit may also include updates to portions of the storage circuit related to a database and/or other data storage format which maintains information for the classification system, such as the signatures, weights, thresholds, etc.

The sample input circuit of the classification system may provide an interface for the platform as described hereinabove to receive biological samples to be analyzed. The sample input circuit may include mechanical elements, as well as electrical elements, which receive a biological sample provided by a user to the classification system and transport the biological sample within the classification system and/or platform to be processed. The sample input circuit may include a bar code reader that identifies a bar-coded container for identification of the sample and/or test order form. The sample processing circuit may further process the biological sample within the classification system and/or platform so as to prepare the biological sample for automated analysis. The sample analysis circuit may automatically analyze the processed biological sample. The sample analysis circuit may be used in measuring, e.g., DNA/gene/CpG island methylation levels of a group/set of genes with the biological sample provided to the classification system. In some embodiments, measuring DNA/methylation levels of a group/set of genes is accomplished on a commercial platform, such as the Illumina Infinium Methylation EPIC BeadChip Kit. In some embodiments, measuring DNA/methylation levels of a group/set of genes is accomplished on custom platforms, such as a customized Illumina Infinium Methylation EPIC BeadChip Kit (EPIC+), and an Illumina Infinium HTS Custom Methylation COVID-19 Panel as described herein. The sample analysis circuit may also retrieve from the storage circuit a classifier for whether a subject infected with SARS-CoV-2, whether a subject is suffering from COVID-19, whether a subject is suffering from ARDS associated with COVID-19 and/or is more likely to suffer from ARDS associated with COVID-19, or whether a subject is suffering from MIS-C associated with COVID-19 and/or is more likely to suffer from MIS-C associated with COVID-19, the classifier(s) include pre-defined weighting values (i.e., coefficients) for each of the gene/DNA methylation sites in the group/set of genes. The sample analysis circuit may enter DNA/gene/CpG island methylation values into one or more classifiers selected from the classifier for whether a subject infected with SARS-CoV-2, whether a subject is suffering from COVID-19, whether a subject is suffering from ARDS associated with COVID-19 and/or is more likely to suffer from ARDS associated with COVID-19, or whether a subject is suffering from MIS-C associated with COVID-19 and/or is more likely to suffer from MIS-C associated with COVID-19. The sample analysis circuit may calculate a probability for one or more of whether the subject has a SARS-CoV-2 infection, whether a subject is suffering from COVID-19, whether a subject is suffering from ARDS associated with COVID-19 and/or is more likely to suffer from ARDS associated with COVID-19, or whether a subject is suffering from MIS-C associated with COVID-19 and/or is more likely to suffer from MIS-C associated with COVID-19 based upon said classifier(s) and control output, via the input/output circuit, of a determination whether an SARS-CoV-2 infection is present or absent, whether a subject is suffering from COVID-19, whether a subject is suffering from ARDS associated with COVID-19 and/or is more likely to suffer from ARDS associated with COVID-19, or whether a subject is suffering from MIS-C associated with COVID-19 and/or is more likely to suffer from MIS-C associated with COVID-19, or some combination thereof. In some embodiments, the sample analysis circuit may calculate a probability or score for the presence of an infection or absence of an infection, such as an infection with SARS-CoV-2, and/or wherein presence of an infection is indicative of a presence of, a likelihood that, and/or a risk that a subject may suffer from ARDS and/or MIS-C.

The sample input circuit, the sample processing circuit, the sample analysis circuit, the input/output circuit, the storage circuit, and/or the update circuit may execute at least partially under the control of the one or more processors of the classification system. As used herein, executing “under the control” of the processor means that the operations performed by the sample input circuit, the sample processing circuit, the sample analysis circuit, the input/output circuit, the storage circuit, and/or the update circuit may be at least partially executed and/or directed by the processor, but does not preclude at least a portion of the operations of those components being separately electrically or mechanically automated. The processor may control the operations of the classification system, as described herein, via the execution of computer program code.

Computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the classification system, partly on the classification system, as a stand-alone software package, partly on the classification system and partly on a remote computer or entirely on the remote computer or server. In the latter scenario, the remote computer may be connected to the classification system through any type of network, including a local area network (LAN) or a wide area network (WAN), or the connection may be made to an external computer (for example, through the Internet using an Internet Service Provider) or in a cloud computer environment or offered as a service such as a Software as a Service (SaaS).

In some embodiments, the system includes computer readable code that can transform quantitative, or semi-quantitative, detection of DNA/gene/CpG island methylation to a cumulative score or probability of the etiology of an infection. In some embodiments, the system includes computer readable code that can transform quantitative, or semi-quantitative, detection of DNA/gene/CpG island methylation to a cumulative score or probability of a presence or absence of an infection, wherein presence of an infection may be indicative of the presence of SARS-CoV-2, whether a subject is suffering from COVID-19, whether a subject is suffering from ARDS associated with COVID-19 and/or is more likely to suffer from ARDS associated with COVID-19, whether a subject is suffering form MIS-A associated with COVID-19 and/or is more likely to suffer from MIS-A associated with COVID-19, or whether a subject is suffering from MIS-C associated with COVID-19 and/or is more likely to suffer from MIS-C associated with COVID-19.

Algorithms used in the methods of the inventive concept may include any machine learning approaches that would be appreciated by one of skill in the art including, for example, linear regression ElasticNet regression, Ridge regression, LASSO regression, support vector machine (SVM) regression, Random Forest® and/or XGBoost decision tree algorithms. In some embodiments, a first machine learning approach may be used to optimize features for a second machine learning approach. For example, SVM training on an initial sample set may be followed by XGBoost decision tree training on further samples in generating a classifier.

In some embodiments, the system is a sample-to-result system, with the components integrated such that a user can simply insert a biological sample to be tested, and a period of time later (preferably a short amount of time, e.g., 10, 30 or 45 minutes, or 1, 2, or 3 hours, up to 8, 12, 24 or 48 hours) receive a result output from the system.

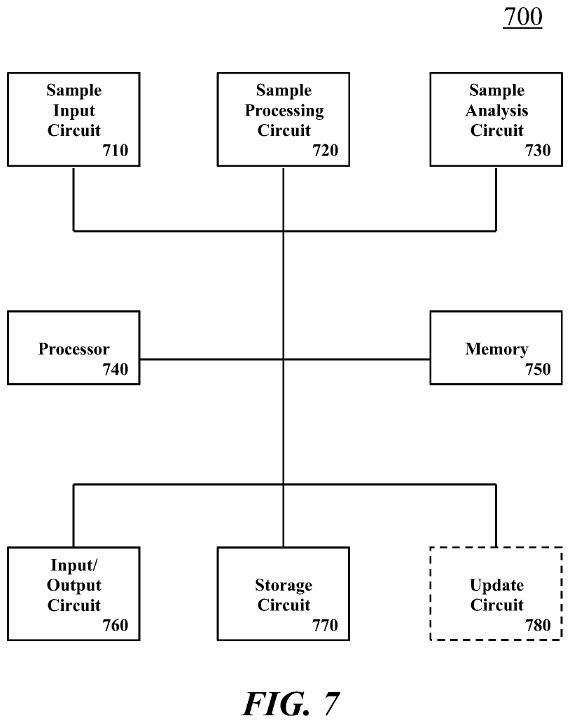

A block diagram of a classification system, computer program product, and/or computer-implemented method that may be used with a platform is depicted in FIG. 7. A classification system 700, computer program product, and/or computer-implemented method may include a processor subsystem 740, including one or more Central Processing Units (CPU) on which one or more operating systems and/or one or more applications run. While one processor 740 is shown, it will be understood that multiple processors 740 may be present, which may be either electrically interconnected or separate. Processor(s) 740 are configured to execute computer program code from memory devices, such as memory 750, to perform at least some of the operations and methods described herein. The storage circuit 770 may store databases which provide access to the data/parameters/classifiers used by the classification system 700 such as the signatures, weights, thresholds, etc. An input/output circuit 760 may include displays and/or user input devices, such as keyboards, touch screens and/or pointing devices. Devices attached to the input/output circuit 760 may be used to provide information to the processor 740 by a user of the classification system 700. Devices attached to the input/output circuit 760 may include networking or communication controllers, input devices (keyboard, a mouse, touch screen, etc.) and output devices (printer or display). An optional update circuit 780 may be included as an interface for providing updates to the classification system 700 such as updates to the code executed by the processor 740 that are stored in the memory 750 and/or the storage circuit 770. Updates provided via the update circuit 780 may also include updates to portions of the storage circuit 770 related to a database and/or other data storage format which maintains information for the classification system 700, such as the signatures, weights, thresholds, etc. The sample input circuit 710 provides an interface for the classification system 700 to receive biological samples to be analyzed. The sample processing circuit 720 may further process the biological sample within the classification system 700 so as to prepare the biological sample for automated analysis by the sample analysis circuit 730. The sample processing circuit 720 and/or sample analysis circuit 730 may operate in conjunction with a platform or technology as described herein, such as, for example, the Illumina Infinium Methylation EPIC BeadChip Kit, the Illumina Infinium Methylation EPIC BeadChip Kit (EPIC+), and the Illumina Infinium HTS Custom Methylation COVID-19 Panel as described herein.

It is to be understood that the invention is not limited in its application to the details of construction and the arrangement of components set forth in the following description or illustrated in the following drawings. The invention is capable of other embodiments and of being practiced or of being carried out in various ways.

Having described various aspects of the inventive concept, the same will be explained in further detail in the following examples, which are included herein for illustrative purposes, and which are not intended to be limiting to the invention.

### Example 1: Epigenetic Hypomethylation and Prediction of Covid-19-Related Outcomes

1. COVID-19 signatures from the Infinium Methylation EPIC BeadChip Kit. Illumina's Infinium Methylation Bead Array has been the workhorse providing the majority of actionable data leveraged in this application, and the latest expansion of the Infinium Methylation technology (i.e., the EPIC BeadChip) provides near epigenome wide coverage (1). The team amassed for the application has considerable expertise in identifying methylation changes associated with respiratory and allergic disease (2-22) and phenotypes associated with viral dissemination (23) leveraging the EPIC platform, through individual research projects and as an institutional service in the Colorado Anschutz Research Genetics Organization (CARGO). Another critical consideration is that the emerging field of epigenetics has demonstrated actionable classification with much smaller sample sizes in contrast to traditional GWAS (24), and the sample size that is proposed (N>3,000) in this application exceeds the power necessary to detect true associations.

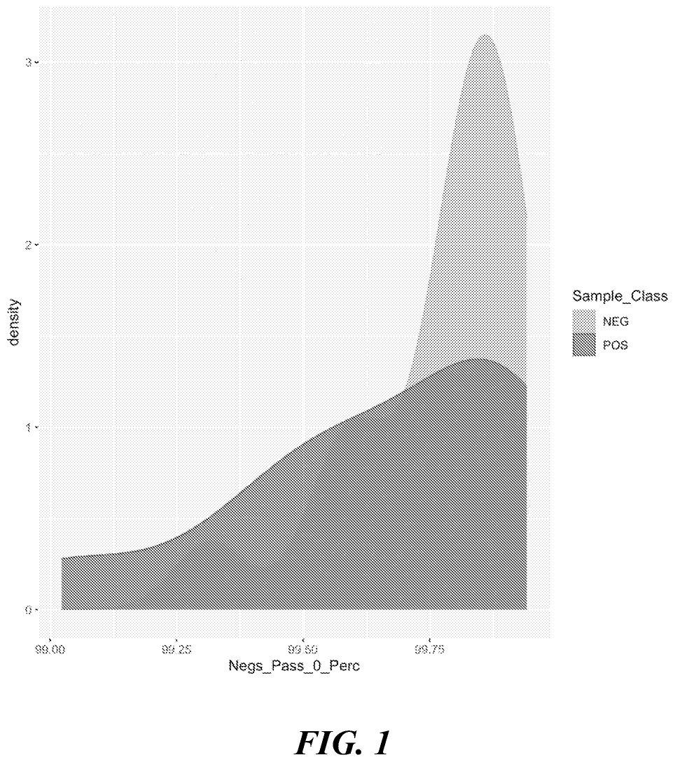

With a goal to leverage EPIC's coverage to classify differential methylation signatures of COVID-19+ and COVID-19− samples, an initial study was completed to demonstrate the ability to accurately distinguish COVID-19+ and COVID-19− DNA samples, and to inform development of a customized Infinium Methylation EPIC BeadChip Kit (referred to as the EPIC Plus) for testing on a larger number of patient samples (Phase 1). The ultimate goal of developing the EPIC Plus chip was to select ˜50K probes for the creation of the Infinium HTS Custom Methylation COVID-19 Panel, the planned platform for the studies in this application (Phase 2). Residual nucleic acid in elution buffer samples from nasopharyngeal swabs (NPS) from 25 patients undergoing rtPCR-based COVID-19 testing in the CCPM Biobank were analyzed for concentration and purity and then bisulfite converted using the EZ DNA Methylation Gold kit (Zymo, Irvine, CA). The bisulfite treated DNA was subjected to whole genome amplification (WGA) via random hexamer priming and Phi29 DNA polymerase, and the amplification products were enzymatically fragmented25, purified from dNTPs, primers, and enzymes, and processed on the Infinium 850K (EPIC) Methylation chip. Overall, via rtPCR, 15 samples tested COVID-19 negative, and 10 COVID-19 positive. The analysis was integrated with existing EPIC NPS methylation data (n=164); as these pre-existing samples were collected pre-COVID-19, they represented appropriate unexposed controls in this proof-of-concept study. With these 15 samples (i) sample process feasibility was demonstrated as quality control filters (implemented in seSAMe (26)) using both negative control probe metrics and seSAMe's p-value-based PooBAH detection thresholds showed >98% call rates, indicating high quality data (FIG. 1). (ii) Using the DML function in seSAMe, 776 sites were identified between COVID-19+ and COVID-19− individuals with delta beta (change in average methylation status). (iii) By increasing the sample size and leveraging 164 pre-COVID-19 controls, the number of delta beta>0.2 probes was increased that could reliably detect between the 2 groups to 25,000. The libsvm package was then used for support vector machine (SVM) (27) training and testing. A two-fold cross validation test was performed to demonstrate clear signal between cases and controls: 3 different SVM classification methods and 3 different detection p-value methods and thresholds were tested (Table 1). Classification accuracy based on 776 CpG sites and 15 samples ranged from 84.6-100. Using 25,000 sites and 164 samples in a L2-regularized L2-loss SVM (dual) classifier and with the negative p-value selection method resulted in 93.7-95.7 accuracy. These results demonstrated very clearly that COVID+ and COVID− samples can be distinguished using epigenetic markers and provided key data for moving forward with the EPIC customization.

2. Development and implementation of a customized Infinium Methylation EPIC BeadChip Kit. Leveraging data generated from NPS samples of COVID-19+ and COVID-19− patients (Section 1 above), in addition to known epigenetic associations with respiratory viral infections and cardiopulmonary complications associated with recent coronavirus outbreaks, all 26,000 known HLA alleles were also targeted, as well as multiple alternative haplotypes and unpublished reference sequences spanning the MHC genomic region, the Natural Killer Cell Immunoreceptor (KIR) and other immunogenetic loci, to enhance the sensitivity of immune response detection and developed a customized Infinium Methylation EPIC BeadChip Kit. The custom ‘EPIC Plus’ chip included ˜10 k sites targeted to increase coverage of the immune response gene panel (Table 2). Table 3 summarizes how the customized chip complements sites already present on the standard EPIC chip. Chips for testing of up to 624 DNA samples were manufactured and provided by Illumina.

Currently, genomic DNA from 312 COVID-19+ and 312 COVID-19− blood samples are being processed, collected from patients recruited through existing protocols at the University of Colorado to (i) confirm the ability to identify an epigenetic signature for SARS-CoV-2 infection and early-stage diagnosis of COVID-19 using genomic DNA from whole blood; and (ii) characterize the epigenetic signature that accurately predicts SARS-CoV-2 infection (confirmed by conventional clinical rtPCR testing and serological data) and select 50K optimal CpG sites to create the Infinium HTS Custom Methylation COVID-19 Panel.

3. Identification of COVID-19 signatures from the EPIC+ chip using blood biospecimens from COVID19 tested patients (Phase 1). Given that genomic DNA from blood is a much more feasible tissue source from suspected COVID-19 cases at scale (largely because it eliminates concerns regarding supply chain issues associated with swabs for the collection of NPS), and because blood has proven a reliable source for generating epigenetic signatures and disease classifiers predicting disease (28-34), the focus was shifted from DNA from NPS samples in the pilot study to blood for Phase 1. To date, results on the first 90 of 624 samples have been generated from the EPIC Plus chip. Identical to the pilot study, DNA samples were analyzed for concentration and purity and then bisulfite converted using the EZ DNA Methylation Gold kit (Zymo, Irvine, CA) as described above, and amplified DNA was processed on the newly developed EPIC Plus methylation chip. Eighty-six samples passed detection p-value cutoffs using the SeSAMe package's PooBAH algorithm, yielding an increased data set of 43 COVID+ and 43 COVID processed over the initial 10 COVID-19+ and 15 COVID-19− controls for training. These samples were split randomly into three groups for three-fold cross validation training and testing using the R implementation of XGBoost (Extreme Gradient Boosting). Table 1 summarizes results from the initial batch of samples in Phase 1. Similar to the pilot study, the Differentially Methylated Loci (DML) function were first applied from SeSAMe package to rank all loci in each random partition. The top N ranked loci were picked for each partition, where N ranged from 100, 1000, 10 k, 50 k, 100 k and 1M (data only shown for 1 k and 10 k). For each bucket training and testing was carried out using XGBoost with default parameters (Table 4).

Besides improving the machine leaning techniques and increasing the sample size beyond the pilot study, in Phase 1 the content of the EPIC array has also been extended by ˜10 k loci to extend host immune response genes. Moreover, a new probe design is included that enables detection and measurement of DNA methylation at multiple specific sites in the human genome. This design type enables epigenetic detection classification in highly homologous regions such as HLA. These new probe designs showed greater than chance selection by the DML algorithm. With the improved machine learning algorithms, increased dataset, improved targeting and novel Infinium probe designs, the strong epigenetic signature between COVID+ and COVID− samples recapitulates in whole blood. It is indicated that the accuracy of this detection is only bounded by the number of samples available to train.

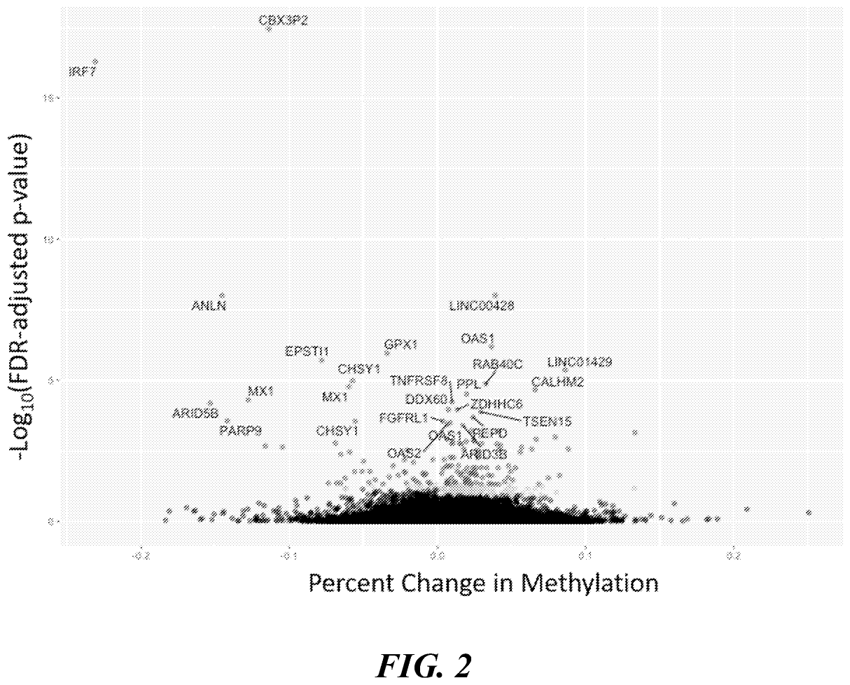

4. Epigenome-wide association study (EWAS) with SARS-CoV-2 infection status. To evaluate epigenome-wide DNA methylation patterns in SARS-CoV-2 infection, peripheral blood was analyzed from data from 43 COVID+ and 43 COVID− individuals described in Section 3 above on the Illumina EPIC Plus array. Illumina idat signal intensity files were processed using seSAMe (26). Probes containing a SNP site (minor allele frequency >1% in the general population) as well as probes with non-unique mapping and off-target hybridization were removed. Additionally, probes with an average detection p value ≥0.05 across samples were removed prior to analysis. This resulted in 748,416 probes that passed quality control and were tested for association with COVID-19 status in an epigenome-wide association analysis (EWAS). Principal component regression analysis (PCRA) was used to identify array position as a strong batch effect and was regressed out using ComBat (35). Differentially methylated CpGs were identified using linear models in Limma (36), adjusting for age, sex, and race/ethnicity. p values were adjusted for inflation and bias using Bacon (37) and for multiple comparisons using the Benjamini-Hochberg False Discovery Rate (FDR) (38). This analysis identified 145 CpGs significant at FDR-adjusted p-value<0.05 (FIG. 2). A CpG within the Interferon Response Factor 7 (IRF7) gene is the most hypomethylated, with an average change in methylation of −23% in the COVID19 positive compared to COVID-19 negative group. Furthermore, enrichment analysis identified multiple interferon-related categories as most enriched within differentially methylated genes (GO Biological Process Cellular Response to Type I Interferon, BioPlanet Pathway Interferon Alpha/Beta Signaling, Jensen Compartments Interferon Regulatory Factor Complex and others). These data suggest hypomethylation and therefore activation of interferon response, as has been previously observed for SARS infections (39,40). Significant differences were not observed in cell proportions in COVID-19 positive and COVID-19 negative groups, as estimated using the Houseman method (41), indicating that the methylation changes we identified are not confounded by cell proportions.

### REFERENCES

1 Pidsley, R. et al. Critical evaluation of the Illumina MethylationEPIC BeadChip microarray for whole-genome DNA methylation profiling. Genome Biol 17, 208, doi:10.1186/s13059-016-1066-1 (2016).
2 Breton, C. V. et al. Prenatal tobacco smoke exposure is associated with childhood DNA CpG methylation. PloS one 9, e99716, doi:10.1371/journal.pone.0099716 (2014).
3 Kothari, P. H. et al. Role of local CpG DNA methylation in mediating the 17q21 asthma susceptibility gasdermin B (GSDMB)/ORMDL sphingolipid biosynthesis regulator 3 (ORMDL3) expression quantitative trait locus. The Journal of allergy and clinical immunology 141, 2282-2286 e2286, doi:10.1016/j.jaci.2017.11.057 (2018).
4 Yang, I. V. et al. Relationship of DNA methylation and gene expression in idiopathic pulmonary fibrosis. American journal of respiratory and critical care medicine 190, 1263-1272, doi:10.1164/rccm.201408-14520C (2014).
5 Eyring, K. R., Pedersen, B. S., Yang, I. V. & Schwartz, D. A. In Utero Cigarette Smoke Affects Allergic Airway Disease But Does Not Alter the Lung Methylome. PloS one 10, e0144087, doi:10.1371/journal.pone.0144087 (2015).
6 Helling, B. A. & Yang, I. V. Epigenetics in lung fibrosis: from pathobiology to treatment perspective. Current opinion in pulmonary medicine 21, 454-462, doi:10.1097/MCP.0000000000000191 (2015).
7 Liang, L. et al. An epigenome-wide association study of total serum immunoglobulin E concentration. Nature 520, 670-674, doi:10.1038/nature14125 (2015).
8 Yang, I. V. et al. DNA methylation and childhood asthma in the inner city. The Journal of allergy and clinical immunology 136, 69-80, doi:10.1016/j.jaci.2015.01.025 (2015).
9 Breton, C. V. et al. Small-Magnitude Effect Sizes in Epigenetic End Points are Important in Children's Environmental Health Studies: The Children's Environmental Health and Disease Prevention Research Center's Epigenetics Working Group. Environmental health perspectives 125, 511-526, doi:10.1289/EHP595 (2017).
10 Helling, B. A. et al. Regulation of MUC5B Expression in Idiopathic Pulmonary Fibrosis. American journal of respiratory cell and molecular biology 57, 91-99, doi:10.1165/rcmb.2017-00460C (2017).
11 Yang, I. V., Lozupone, C. A. & Schwartz, D. A. The environment, epigenome, and asthma. The Journal of allergy and clinical immunology 140, 14-23, doi:10.1016/j.jaci0.2017.05.011 (2017).
12 Yang, I. V. et al. The nasal methylome and childhood atopic asthma. Journal of Allergy and Clinical Immunology 139, 1478-1488, doi:10.1016/j.jaci.2016.07.036 (2017).
13 Yang, I. V. et al. The Nasal Methylome: A Key to Understanding Allergic Asthma. American journal of respiratory and critical care medicine 195, 829-831, doi:10.1164/rccm.201608-1558LE (2017).
14 Davidson, E. J. & Yang, I. V. Role of epigenetics in the development of childhood asthma. Current opinion in allergy and clinical immunology 18, 132-138, doi:10.1097/ACI.0000000000000429 (2018).
15 Eyring, K. R. et al. Methylene-tetrahydrofolate reductase contributes to allergic airway disease. PloS one 13, e0190916, doi:10.1371/journal.pone.0190916 (2018).
16 Yang, I. V. DNA methylation signatures of atopy and asthma. Lancet Respir Med, doi:10.1016/S2213 2600(18)30504-6 (2018).
17 Reese, S. E. et al. Epigenome-wide meta-analysis of DNA methylation and childhood asthma. The Journal of allergy and clinical immunology 143, 2062-2074, doi:10.1016/j.jaci.2018.11.043 (2019).
18 Yang, I. V. et al. DNA Methylation Changes in Lung Immune Cells Are Associated with Granulomatous Lung Disease. American journal of respiratory cell and molecular biology 60, 96-105, doi:10.1165/rcmb.2018-01770C (2019).
19 Qi, C. et al. Nasal DNA methylation profiling of asthma and rhinitis. The Journal of allergy and clinical immunology, doi:10.1016/j.jaci.2019.12.911 (2020).
20 Yang I V et al. DNA Methylation Changes and Childhood Asthma in the Inner City J Allergy Clin Immunol. accepted (2015).
21 Forno, E. et al. DNA methylation in nasal epithelium, atopy, and atopic asthma in children: a genomewide study. Lancet Respir Med, doi:10.1016/S2213-2600(18)30466-1 (2018).
22 Cardenas, A. et al. The nasal methylome as a biomarker of asthma and airway inflammation in children. Nature communications 10, 3095, doi:10.1038/s41467-019-11058-3 (2019).
23 Boorgula, M. P. et al. Replicated methylation changes associated with eczema herpeticum and allergic response. Clinical epigenetics 11, 122, doi:10.1186/s13148-019-0714-1 (2019).
24 Tsai, P. C. & Bell, J. T. Power and sample size estimation for epigenome-wide association scans to detect differential DNA methylation. Int J Epidemiol 44, 1429-1441, doi:10.1093/ije/dyv041 (2015).
25 Weisenberger D J, V. D. B. D., Pan F, Berman B P, and Laird P W. Comprehensive DNA Methylation Analysis on the Illumina Infinium Assay Platform. Illumina application note (2008).
26 Zhou, W., Triche, T. J., Jr., Laird, P. W. & Shen, H. SeSAMe: reducing artifactual detection of DNA methylation by Infinium BeadChips in genomic deletions. Nucleic Acids Res 46, e123, doi:10.1093/nar/gky691 (2018).
27 Chang, C.-C. & Lin, C.-J. LIBSVM: a library for support vector machines. ACM Transactions on Intelligent Systems and Technology 2:27, 1-27 (2011).
28 Jiang, H. et al. DNA methylation markers in the diagnosis and prognosis of common leukemias. Signal Transduct Target Ther 5, 3, doi:10.1038/s41392-019-0090-5 (2020).
29 Ciolfi, A. et al. Frameshift mutations at the C-terminus of HIST1H1E result in a specific DNA hypomethylation signature. Clin Epigenetics 12, 7, doi:10.1186/s13148-019-0804-0 (2020).
30 Wang, L., Ni, S., Du, Z. & Li, X. A six-CpG-based methylation markers for the diagnosis of ovarian cancer in blood. J Cell Biochem 121, 1409-1419, doi:10.1002/jcb.29376 (2020).
31 Imgenberg-Kreuz, J. et al. Shared and Unique Patterns of DNA Methylation in Systemic Lupus Erythematosus and Primary Sjogren's Syndrome. Front Immunol 10, 1686, doi:10.3389/fimmu.2019.01686 (2019).
32 Bend, E. G. et al. Gene domain-specific DNA methylation episignatures highlight distinct molecular entities of ADNP syndrome. Clin Epigenetics 11, 64, doi:10.1186/s13148-019-0658-5 (2019).
33 Wang, C., Chen, L., Yang, Y., Zhang, M. & Wong, G. Identification of potential blood biomarkers for Parkinson's disease by gene expression and DNA methylation data integration analysis. Clin Epigenetics 11, 24, doi:10.1186/s13148-019-0621-5 (2019).
34 Panagopoulou, M. et al. Circulating cell-free DNA in breast cancer: size profiling, levels, and methylation patterns lead to prognostic and predictive classifiers. Oncogene 38, 3387-3401, doi:10.1038/s41388-018-0660-y (2019).
35 Leek, J. T., Johnson, W. E., Parker, H. S., Jaffe, A. E. & Storey, J. D. The sva package for removing batch effects and other unwanted variation in high-throughput experiments. Bioinformatics 28, 882-883, doi:10.1093/bioinformatics/bts034 (2012).
36 Ritchie, M. E. et al. limma powers differential expression analyses for RNA-sequencing and microarray studies. Nucleic acids research 43, e47, doi:10.1093/nar/gkv007 (2015).
37 van Iterson, M., van Zwet, E. W., Consortium, B. & Heijmans, B. T. Controlling bias and inflation in epigenome- and transcriptome-wide association studies using the empirical null distribution. Genome biology 18, 19, doi:10.1186/s13059-016-1131-9 (2017).
38 Benjamini, Y. & Hochberg, Y. Controlling the False Discovery Rate: A Practical and Powerful Approach to Multiple Testing. Journal of the Royal Statistical Society. Series B (Methodological) 57, 289-300 (1995).
39 Totura, A. L. & Baric, R. S. SARS coronavirus pathogenesis: host innate immune responses and viral antagonism of interferon. Curr Opin Virol 2, 264-275, doi:10.1016/j.coviro.2012.04.004 (2012).
40 Menachery, V. D. et al. Pathogenic influenza viruses and coronaviruses utilize similar and contrasting approaches to control interferon-stimulated gene responses. mBio 5, e01174-01114, doi:10.1128/mBio.01174-14 (2014).
41 Houseman, E. A. et al. DNA methylation arrays as surrogate measures of cell mixture distribution. BMC bioinformatics 13, 86, doi:10.1186/1471-2105-13-86 (2012).

### Example 2: Potential Role for Epigenetic Signatures in the Clinical Presentation of Multisystem Inflammatory Syndrome in Children (MIS-C) Associated with Covid-19

Enveloped RNA viruses (e.g., coronavirus) manipulate the host's epigenome by antagonizing & regulating the host innate immune antiviral defense processes, specifically via DNA methylation. Epigenetic modification has been reported in other viral infections (influenza, respiratory syncytial virus, rhinovirus, adenovirus), respiratory (i.e., asthma, COPD) & CVD, complications of COVID-19. Epigenetic changes may predict worsening of cardio-respiratory complications associated with COVID-19 infection (i.e., ARDS), and multisystem inflammatory syndrome in children (MIS-C) associated with COVID-19.

The German Cancer Research Center (DKFZ) deployed DNA methylation-based diagnosis for CNS tumor diagnostics. Unsupervised clustering of DNA methylation array data for >90 CNS tumor types showed that distinct tumors are well-classified based on their epigenetic signatures. Using the Illumina EPIC methylation array, the DKFZ created a web-distributed random forest classifier to accurately diagnose CNS tumor type. The classifier reduced tumor misclassification by ˜15% (see, Sawahla et al. DNA methylation-based classification of central nervous system tumors. Nature 555, 469, doi:10.1038/nature26000).

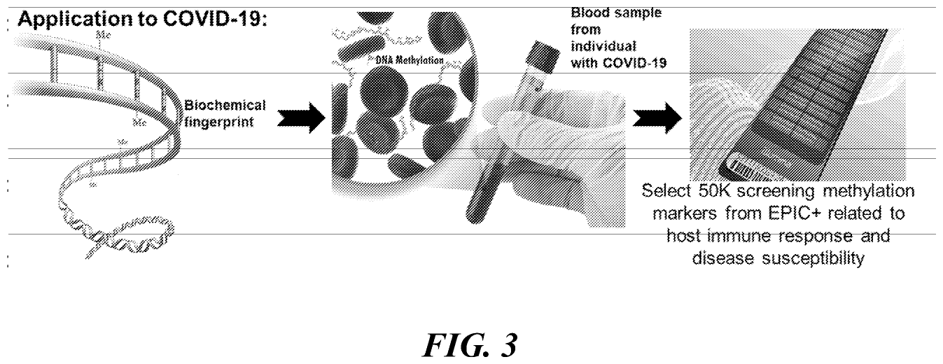

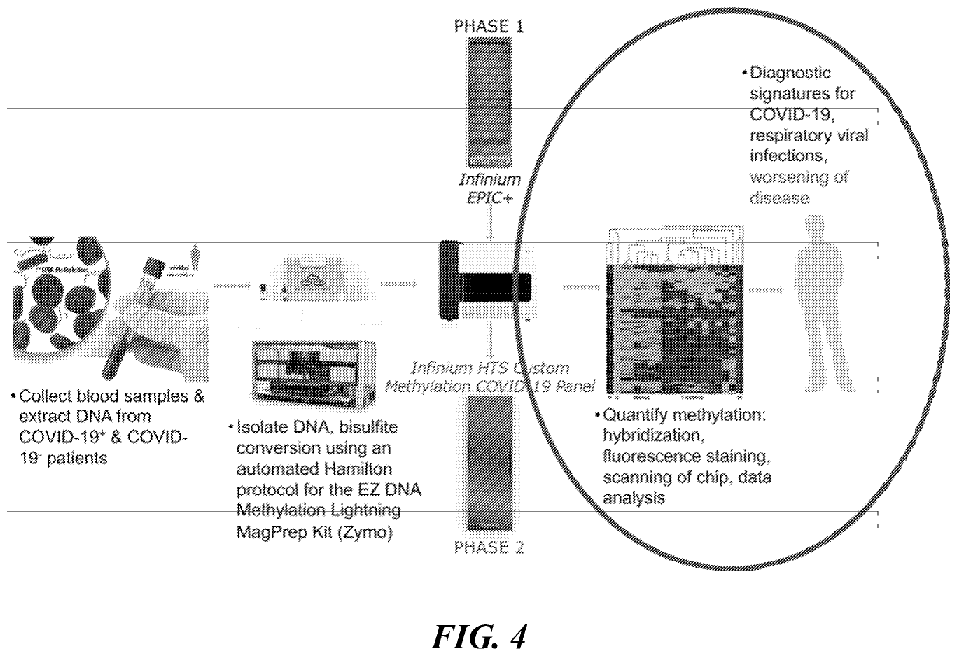

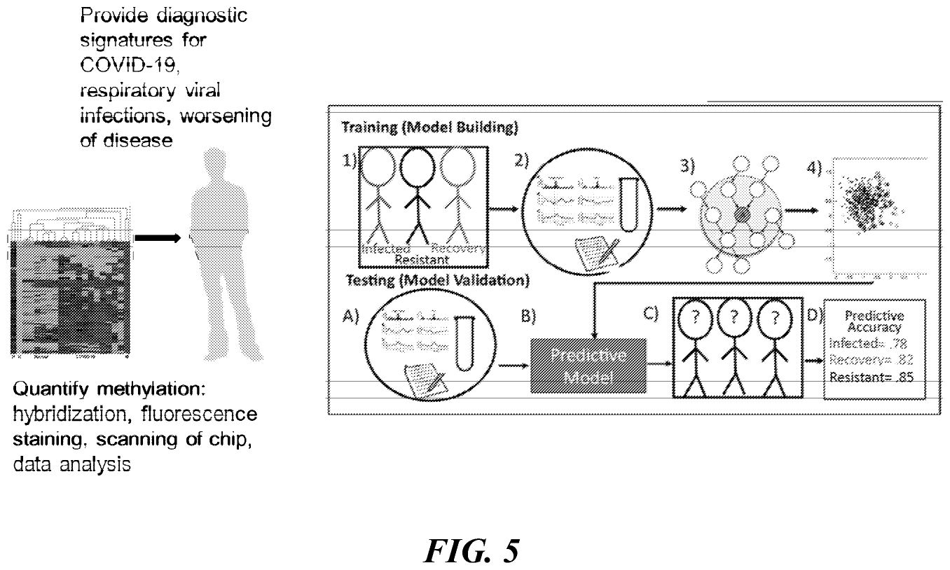

For application methylation-based classification to COVID-19 and complications associated with COVID-19, DNA methylation patterns are analyzed in DNA extracted from blood samples from COVID-19+ and COVID-19− patients (FIG. 3). The isolated DNA samples are analyzed for concentration and purity and subjected to bisulfite conversion using an automated Hamilton protocol for the EZ DNA Methylation Lightning MagPrep kit (ZYMO, Irvine, CA). The bisulfite-converted DNA is subjected to amplification and the amplified DNA processed on the newly developed Infinium EPIC Plus methylation chip (Phase 1) and on an Infinium HTS custom methylation COVID-19 panel (Phase 2). Methylation is quantified through hybridization, fluorescence staining, chip scanning, and data analysis, and diagnostic signatures provided for COVID-19, respiratory viral infections, and worsening of disease, for example, diagnostic signatures can be provided through analysis using samples from children with COVID-19 and/or MIS-C (FIG. 4). Exemplary SVM training and testing on COVID-19+ cases and COVID-19− controls is depicted in FIG. 5 and summarized in Table 1 of EXAMPLE 1.

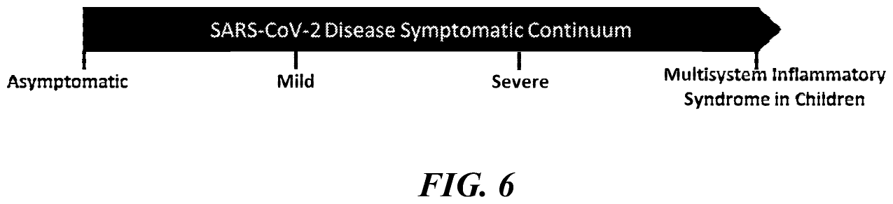

The Infinium HTS custom methylation COVID-19 panel can be used to assess disease state within the SARS-CoV-2 disease symptomatic continuum shown in FIG. 6, from asymptomatic, to mild, to severe, and to MIS-C. The custom panel provides high coverage and accuracy at a low cost (less than about $100/sample) with results in about 48 hours. It can be widely deployed, as it runs on, for example, the Illumina Infinium platform in research and clinical laboratories, as well as devices, such as NextSeq550, found in and routinely available in smaller clinical laboratories. Maximum throughput possible is shown in Table 5. Requirements per test is about 500 ng genomic DNA that can be collected in, for example, a dedicated 4 mL aliquot of EDTA (average yield=about 50 ng/μL, equivalent to ˜7.5 μL).

### Example 3: Analysis Pipeline and Machine Learning Approaches

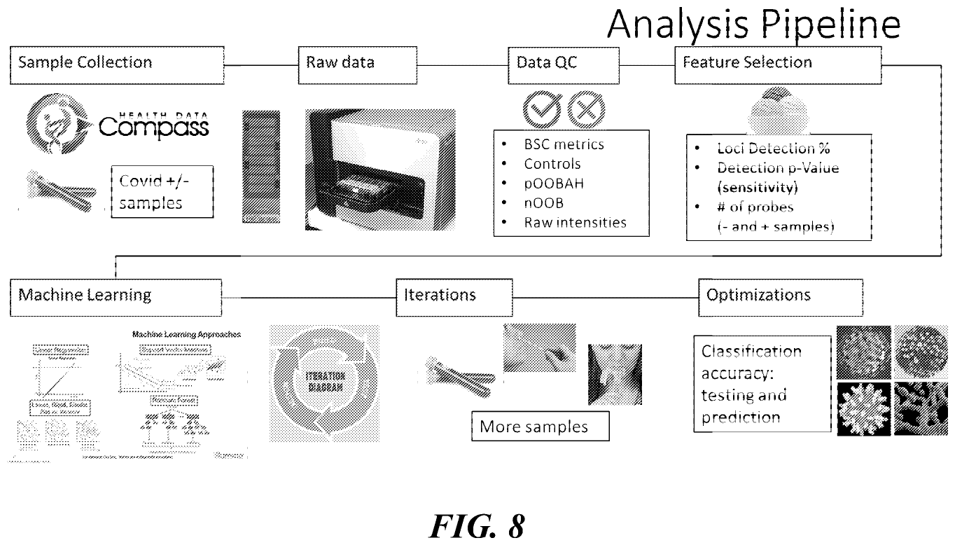

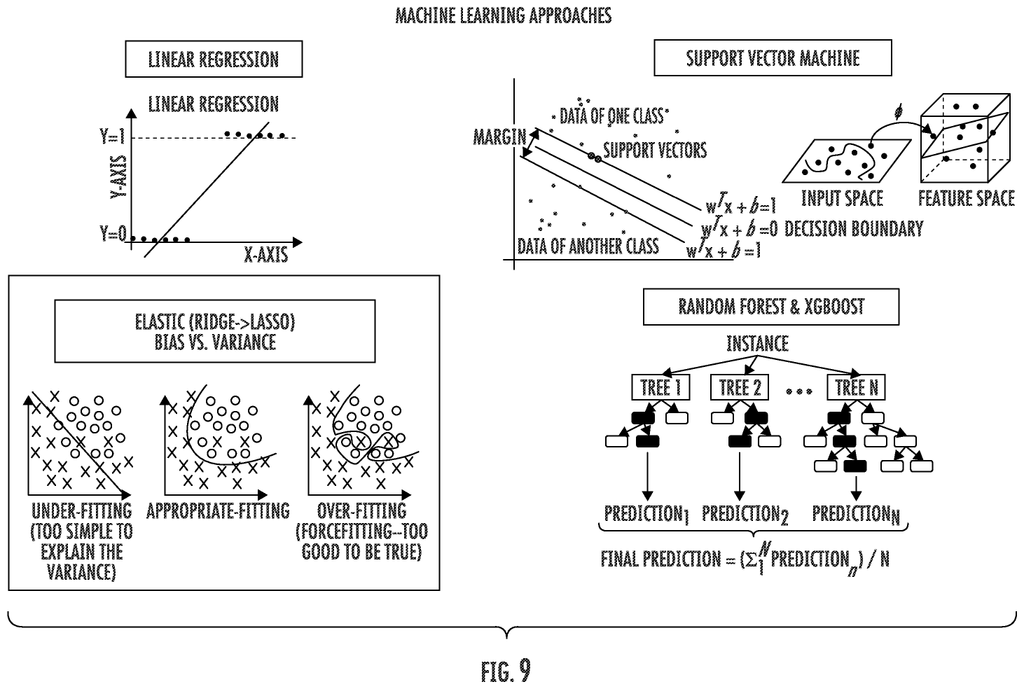

Classifiers for assessing disease state is accomplished by collecting data for DNA methylation from samples of known COVID-19 status (COVID-19+ and COVID-19−). The raw data is subjected to QC/normalization using BSC metrics, controls, pOOBAH, and nOOB background correction. Upfront QC is made based on loci detection percentage, detection p-value (sensitivity) and number of probes (+ and − samples). The signature is subjected to supervised machine learning and a classifier generated through iteration with more samples and adjusting weighting of features until accuracy of classification is optimized. An outline of classifier generation is depicted in FIG. 8. Approaches to machine learning are depicted in FIG. 9. Algorithms for machine learning may include linear regression, ElasticNet regression, Ridge regression, LASSO regression, support vector machine (SVM) regression, Random Forest® and XGBoost decision tree algorithms. Results from cross-validation using SVMs as a first analysis, is shown in Table 6. SVM training and testing on COVID-19+ NPS samples and COVID-19− controls, followed by XGBoost training and testing on COVID-19+ and COVID-19− blood samples. From this analysis, it can be concluded that there is a difference in DNA methylation signature between COVID-19+ and COVID-19− samples.

One skilled in the art will readily appreciate that the present invention is well adapted to carry out the objects and obtain the ends and advantages mentioned, as well as those inherent therein. The present disclosures described herein are presently representative of particular embodiments, are exemplary, and are not intended as limitations on the scope of the invention. Changes therein and other uses will occur to those skilled in the art which are encompassed within the spirit of the invention as defined by the scope of the claims.

TABLE 1
SVM training and testing on the initial 10 COVID-19+ NPS samples plus 164 COVID− controls
(pilot study), followed by XGBoost training and testing on 43 COVID-19+ and 43 COVID-19− blood samples.

| | PooBAH = 0.1 | | PooBAH = 0.2 | | Negative = 0.05 | | | | | | |
|---|---|---|---|---|---|---|---|---|---|---|---|
| | Cross- | Cross- | Cross- | Cross- | Cross- | Cross- | | | | | |
| | valid. 1 | valid. 2 | valid. 1 | valid. 2 | valid. 1 | valid. 2 | COVID+ | COVID+ | COVID+ | COVID+ | # of |
| | accuracy | accuracy | accuracy | accuracy | accuracy | accuracy | partition A | partition A | partition B | partition B | loci |
| L2- | 84.6154 | 91.6667 | 100 | 91.6667 | 100 | 91.6667 | 5 | 8 | 5 | 7 | 776 |
| regularized | | | | | | | | | | | |
| L2 loss SVM | | | | | | | | | | | |
| (dual) | | | | | | | | | | | |
| L2- | 84.6154 | 91.6667 | 92.3077 | 91.6667 | 100 | 91.6667 | 5 | 8 | 5 | 7 | 776 |
| regularized | | | | | | | | | | | |
| L2 loss SVM | | | | | | | | | | | |
| (primal) | | | | | | | | | | | |
| L2- | 84.6154 | 91.6667 | 100 | 91.6667 | 100 | 91.6667 | 5 | 8 | 5 | 7 | 776 |
| regularized | | | | | | | | | | | |
| L1 loss SVM | | | | | | | | | | | |
| (dual) | | | | | | | | | | | |
| XGBoost- | | | | | 99.4 | 98.8 | 43 | 43 | 43 | 43 | 1000 |
| multi: | | | | | | | | | | | |
| softprob- | | | | | | | | | | | |
| mlogloss | | | | | | | | | | | |
| XGBoost- | | | | | 99.4 | 97.1 | 43 | 43 | 43 | 43 | 50000 |
| multi: | | | | | | | | | | | |
| softprob- | | | | | | | | | | | |
| mlogloss | | | | | | | | | | | |

TABLE 2
Methylation coverage before and after customization.

| | | | | Probes/ | Probes/ |
|---|---|---|---|---|---|
| | Genes | Transcripts | Probes | gene | transcript |
| EPIC | 303 | 1,453 | 12,066 | 39.82 | 8.30 |
| EPIC+ | 387 | 1,598 | 62,066 | 160.38 | 38.84 |

TABLE 3
EPIC genome-wide coverage and immune-specific coverage (transcripts
& genes) before and after customization (build = hg38).

| | | | | Whole | | | Immune | |
|---|---|---|---|---|---|---|---|---|
| | | | | Genome | | | Response | |
| | Genomic | Genomic | | Average | Median | | Average | Median |
| | Feature | Class | Total | Probes | Probes | Total | Probes | Probes |
| EPIC | Transcript | Body* | 88,165 | 16.8 | 10 | 1,333 | 22.2 | 13 |
| | | TSS1500** | 83,411 | 5 | 4 | 1,606 | 6.8 | 4 |
| | | TSS200† | 70,149 | 2.7 | 2 | 1,262 | 2.8 | 2 |
| | Gene | Body* | 18,586 | 20.1 | 11 | 259 | 27.7 | 16 |
| | | TSS1500** | 20,992 | 7.3 | 5 | 281 | 16.1 | 7 |
| | | TSS200† | 16,530 | 5.4 | 4 | 237 | 7.9 | 5 |
| EPIC+ | Transcript | Body* | 5,935 | 7.3 | 1 | 966 | 36.3 | 22.5 |
| | | TSS1500** | 1,299 | 12.6 | 10 | 1,135 | 14.2 | 12 |
| | | TSS200† | 852 | 4.5 | 3 | 844 | 4.5 | 3 |
| | Gene | Body* | 1,387 | 6.5 | 1 | 163 | 42.4 | 23 |
| | | TSS1500** | 331 | 21.5 | 8 | 218 | 31.6 | 16 |
| | | TSS200† | 175 | 11.8 | 7 | 169 | 12.2 | 7 |

*Body = the whole gene body;
**TSS1500-201-1500 base pairs upstream of the transcription start site for each transcript;
†TSS200 = 1-200 base pairs upstream of the transcription start site for each transcript.

TABLE 4
XGBoost training and testing on 43 COVID+ cases and 43 COVID− controls.

| | Negative = 0.05 | | COVID+ | COVID− | COVID+ | COVID− | |
|---|---|---|---|---|---|---|---|
| | Cross-valid. 1 | Cross-valid. 2 | samples | samples | samples | samples | Number |
| | accuracy | accuracy | partition A | partition A | partition B | partition B | of loci |
| L2-regularized | 93.6842 | 95.7447 | 5 | 90 | 5 | 89 | 10000 |
| L2 loss SVM | | | | | | | |
| (dual) | | | | | | | |
| L2-regularized | 94.7368 | 95.7447 | 5 | 90 | 5 | 89 | 10000 |
| L2 loss SVM | | | | | | | |
| (primal) | | | | | | | |

TABLE 5
Maximum throughput for Illumina Infinium
HTS custom methylation COVID-19 panel

| Maximum | Monthly per | Yearly per | Monthly per | Yearly per |
|---|---|---|---|---|
| Throughput | 2 iScan | 2 iScan | 4 iScan | 4 iScan |
| Samples | 30,400 | 364,800 | 60,000 | 720,000 |

TABLE 6
SVM training and testing on the known COVID-19+ samples and COVID−
controls, followed by training and testing on COVID-19+ cases and COVID-19− controls.

| | PooBAH = 0.1 | | PooBAH = 0.2 | | Negative = 0.05 | | | | | | |
|---|---|---|---|---|---|---|---|---|---|---|---|
| | Cross- | Cross- | Cross- | Cross- | Cross- | Cross- | | | | | |
| | valid. 1 | valid. 2 | valid. 1 | valid. 2 | valid. 1 | valid. 2 | COVID+ | COVID+ | COVID+ | COVID+ | # of |
| | accuracy | accuracy | accuracy | accuracy | accuracy | accuracy | partition A | partition A | partition B | partition B | loci |
| L2- | 84.6154 | 91.6667 | 100 | 91.6667 | 100 | 91.6667 | 5 | 8 | 5 | 7 | 776 |
| regularized | | | | | | | | | | | |
| L2 loss SVM | | | | | | | | | | | |
| (dual) | | | | | | | | | | | |
| L2- | 84.6154 | 91.6667 | 92.3077 | 91.6667 | 100 | 91.6667 | 5 | 8 | 5 | 7 | 776 |
| regularized | | | | | | | | | | | |
| L2 loss SVM | | | | | | | | | | | |
| (primal) | | | | | | | | | | | |
| L2- | 84.6154 | 91.6667 | 100 | 91.6667 | 100 | 91.6667 | 5 | 8 | 5 | 7 | 776 |
| regularized | | | | | | | | | | | |
| L1 loss SVM | | | | | | | | | | | |
| (dual) | | | | | | | | | | | |
| L2- | | | | | 93.6482 | 95.7447 | 5 | 90 | 5 | 89 | 10000 |
| regularized | | | | | | | | | | | |
| L2 loss SVM | | | | | | | | | | | |
| (primal) | | | | | | | | | | | |
| L2- | | | | | 94.7368 | 95.7447 | 5 | 90 | 5 | 89 | 10000 |
| regularized | | | | | | | | | | | |
| L1 loss SVM | | | | | | | | | | | |
| (dual) | | | | | | | | | | | |

1. A method of assaying for presence of a viral infection or determining the nature of a viral infection in a subject, the method comprising:
obtaining a methylation pattern for a set of DNA methylation sites from a sample derived from the subject; and
analyzing the methylation pattern for the set of DNA methylation sites with an infection positive methylation classifier and/or an infection negative methylation classifier,
wherein presence of a viral infection in the subject is indicated when a score derived from the infection positive classifier exceeds a cutoff and/or threshold value indicating the presence of a viral infection.

2. The method of claim 1, wherein the viral infection is a SARS-CoV-2 infection.

3. The method of claim 2, wherein the SARS-CoV-2 infection causes COVID-19.

4. (canceled)

5. (canceled)

6. The method of claim 1, wherein obtaining the methylation pattern for the set of DNA methylation sites is performed on a methylation bead array.

7. The method of claim 6, wherein the methylation bead array comprises a set of genes comprising ANLN, ARID3B, ARID5B, CALHM2, CBX3B2, CD38, CHSY1 CMPK2, DDX60, DTX3L, EPSTI1, FAM38A, FGFRL1, GPX1, GTPBP2, IFI27, IFIT3, IRF7, LINC00428, LINC01429, MX1, OAS1, OAS2, PARP9, PHOSPHO1, PPL, RAB40C, REPD, TNFRSF8, TRIM22, TSEN15, and ZDHHC6, or any subset thereof.

8. The method of claim 1, wherein analyzing the methylation pattern for the set of DNA methylation sites with an infection positive methylation classifier and/or an infection negative methylation classifier comprises analyzing methylation of a set of genes comprising ANLN, ARID3B, ARID5B, CALHM2, CBX3B2, CD38, CHSY1 CMPK2, DDX60, DTX3L, EPSTI1, FAM38A, FGFRL1, GPX1, GTPBP2, IFI27, IFIT3, IRF7, LINC00428, LINC01429, MX1, OAS1, OAS2, PARP9, PHOSPHO1, PPL, RAB40C, REPD, TNFRSF8, TRIM22, TSEN15, and ZDHHC6, or any subset thereof.

9. The method of claim 8, wherein an infection positive methylation signature comprises hypomethylation of one or more genes selected from a group consisting of IFR7, ARID5B, ANLN, PARP9, MX1, CBX3P2, EPSTI1, CHSY1, MX1, and GPX1, or any subset thereof, and/or hypermethylation of one or more genes selected from a group consisting of LINC01429, CALHM2, LINC00428, OAS1, RAB40C, TSEN15, PEPD, PPL, ARID3B, ZDHHC6, TNFRSF8, DDX60, OAS2, and FGFRL1, or any subset thereof.

10. The method of claim 9, wherein the infection positive methylation signature comprises hypomethylation of IFR7.

11. A method for determining likelihood of a viral infection producing acute respiratory distress syndrome in a subject comprising:
obtaining a methylation pattern for a set of DNA methylation sites from a sample derived from the subject; and
analyzing the methylation pattern for the set of DNA methylation sites with an acute respiratory distress syndrome positive methylation classifier and/or an acute respiratory distress syndrome negative methylation classifier,
wherein likelihood the subject will exhibit acute respiratory distress syndrome is indicated when a score derived from the acute respiratory distress syndrome positive classifier exceeds a cutoff and/or threshold value indicating likelihood of a viral infection producing acute respiratory distress syndrome.

12. The method of claim 11, wherein the viral infection is a SARS-CoV-2 infection.

13. The method of claim 12, wherein the SARS-CoV-2 infection causes COVID-19.

14. (canceled)

15. (canceled)

16. The method of claim 11, wherein obtaining the methylation pattern for the set of DNA methylation sites is performed on a methylation bead array.

17. The method of claim 16, wherein the methylation bead array comprises a set of genes comprising ANLN, ARID3B, ARID5B, CALHM2, CBX3B2, CD38, CHSY1 CMPK2, DDX60, DTX3L, EPSTI1, FAM38A, FGFRL1, GPX1, GTPBP2, IFI27, IFIT3, IRF7, LINC00428, LINC01429, MX1, OAS1, OAS2, PARP9, PHOSPHO1, PPL, RAB40C, REPD, TNFRSF8, TRIM22, TSEN15, and ZDHHC6, or any subset thereof.

18. The method of claim 11, wherein analyzing the methylation pattern for the set of DNA methylation sites with an infection positive methylation classifier and/or an infection negative methylation classifier comprises analyzing methylation of a set of genes comprising ANLN, ARID3B, ARID5B, CALHM2, CBX3B2, CD38, CHSY1 CMPK2, DDX60, DTX3L, EPSTI1, FAM38A, FGFRL1, GPX1, GTPBP2, IFI27, IFIT3, IRF7, LINC00428, LINC01429, MX1, OAS1, OAS2, PARP9, PHOSPHO1, PPL, RAB40C, REPD, TNFRSF8, TRIM22, TSEN15, and ZDHHC6, or any subset thereof.

19. The method of claim 18, wherein an acute respiratory distress syndrome positive methylation signature comprises hypomethylation of one or more genes selected from a group consisting of IFR7, ARID5B, ANLN, PARP9, MX1, CBX3P2, EPSTI1, CHSY1, MX1, and GPX1, or any subset thereof, and/or hypermethylation of one or more genes selected from a group consisting of LINC01429, CALHM2, LINC00428, OAS1, RAB40C, TSEN15, PEPD, PPL, ARID3B, ZDHHC6, TNFRSF8, DDX60, OAS2, and FGFRL1, or any subset thereof.

20. The method of claim 19, wherein the infection positive methylation signature comprises hypomethylation of IFR7.

21-28. (canceled)

29. A computer-implemented method for executing the method of claim 1.

30. (canceled)

31. A method of treating a disorder selected from COVID-19, acute respiratory distress syndrome (ARDS), or multisystem inflammatory syndrome in children (MIS-C), in a subject comprising:
obtaining a methylation pattern for a set of DNA methylation sites from a sample derived from the subject suspected of having the disorder;
analyzing the methylation pattern for the following:
(a) the set of DNA methylation sites with a SARS-CoV-2 positive methylation classifier and/or a SARS-CoV-2 negative methylation classifier, wherein presence of a SARS-CoV-2 infection in the subject is indicated when a score derived from the SARS-CoV-2 positive methylation classifier exceeds a cutoff and/or threshold value indicating presence of a SARS-CoV-2 infection, and treating the subject for COVID-19 if the presence of a SARS-CoV-2 infection in the subject is indicated;
(b) the set of DNA methylation sites to an ARDS positive methylation classifier and/or an ARDS negative methylation classifier, wherein likelihood the subject will exhibit acute respiratory distress syndrome is indicated when a score derived from the ARDS positive classifier exceeds a cutoff and/or threshold value indicating likelihood of a COVID-19 infection producing ARDS, and treating the subject for ARDS; or
(c) the set of DNA methylation sites to an MIS-C positive methylation classifier and/or an MIS-C negative methylation classifier, wherein likelihood the subject will exhibit MIS-C is indicated when a score derived from the MIS-C positive classifier exceeds a cutoff and/or threshold value indicating likelihood of a COVID-19 infection producing MIS-C, and treating the subject for MIS-C.

32-45. (canceled)

46. The method of claim 31, wherein the ARDS positive methylation signature comprises hypomethylation of IFR7.

47-55. (canceled)

56. An array for detecting the presence of a respiratory virus in a sample or a clinical outcome of a respiratory viral infection in a patient comprising a plurality of oligonucleotide probes attached to a solid support; wherein the plurality of oligonucleotide probes detect a methylation signature of the sample or from the patient.

57. (canceled)

58. The array of claim 56, wherein the methylation signature comprises detecting a methylation status of at least one gene selected from the group consisting of: A_24_P561165, AA455656, ABCF1, ACBD5, AGL, AGPAT1, AID, AIF1, AIM2, ANLN, ANKRD28, APE1, APOBEC3G, APOL2, APOL3, APOL6, APPBP2, ARID3B, ARID5B, ASK1, ASPM, ATAT1, ATF2, ATP2C1, B2M, BATF, BATF2, BCL2L14, BRD2, C10orf81, C1orf68, C1R, C1S, C2, C3, C4A, C4B, C5, C6, C6orf136, C6orf15, C7, C8, C9, CALHM2, CALR, CASP1, Caspase-3, Caspase-8, CATSPER2, CATSPER2P1, CATSPERG, CBX3B2, CCDC66, CCHCR1, CCRL1, CD19, CD27-AS1, CD4, CD38, CD40, CD74, CD8, CDKN1A, CELF4, CEP162, CFB, CFH, CFHR1, CFHR2, CFHR3, CFHR4, CHSY1, CHTOP, CIITA, CIITA BX117479, c-Jun, CLIC5, CMPK2, CSNK2B, CTNND1, CTSS ZBP1, CUTA, CX3CL1 A_24_P912985, CXCL10, CXCL11, CXCL2, CYP21A2, DAG1, DDR1, DDX60, DENND2B, DEXI, DTNB, DTX3L, DYSF, E2F5, EGFL8, EHMT2, eIF-2, eIF2B, ELAVL2, EPSTI1, ERP27, ETV7, FADD, FAM26F, FAM38A, FAM49B, FGFRL1 GPX1, FKBP5, FLG, FLG-AS1, FRMD3, FZD5, GAPVD1, GPANK1, GPX5, GTPBP2, GTF2H4, HCG17, HCG18, HCG20, HCG22, HCG24, HCG25, HCG27, HCG4, HCG4B, HCP5, HCP5 NNMT, HLA-A, HLA-B, HLA-C, HLA-DMB, HLA-DOA, HLA-DPA1, HLA-DPB1, HLA-DPB2, HLA-DQA1, HLA-DQA2, HLA-DQB1, HLA-DQB1-AS1, HLA-DRA, HLA-DRB3, HLA-DRB4, HLA-DRB5, HLA-E, HLA-F, HLA-F-AS1, HLA-G, HLA-H, HLA-V, HRNR, HSD17B8, HSPA1B, ICAM1, IFI16, IFI27, IFI35, IFI44, IFI44L, IFIH1, IFIT2, IFIT3, IFIT5, IFITM1, IFNL1, IFNL2, IFNL3, IFNL4, IFN-α, IFN-β, IFNγ, IFN-γ, IFN-ε, IFN-κ, IFN-ω, IGH, IGK, IGL, IKK-α, IKK-β, IKK-γ, IKK-ε, IL-10, IL-11, IL-12, IL12A, IL-13, IL-15, IL-17, IL-18, IL18BP, IL-1ra, IL-1α, IL-1β, IL-2, IL-3, IL-33, IL-36ra, IL-36α, IL-36β, IL-36γ, IL-37, IL-38, IL-4, IL-5, IL-6, IL-7, IL-8, IL-9, IRF1, IRF3, IRF7, IVL, JAK2, JNK, KCTD16, KDM4C, KIFC1, KPRP, LAP3, LCE1D, LCE1E, LCE2B, LCE2C, LCE2D, LCE3C, LCE4A, LELP1, LGP2, LINC00302, LINC00428, LINC01429, LINC01185, LOC100129636, LOC100294145, LOC100507547, LOC101929006, LOC105375690, LRBA, LST1, LTA, LTBR, LT-α, LY6G6F-LY6G6D, LYRM4-AS1, M27126, MAVS, MCCD1, MDA5, MDC1, MEF2C, MEKK1, MICA, MICA-AS1, MICAB, MICB, MICB-DT, MIR219A1, MIR4479, MKK7, MMP25, MOG, MPIG6B, MSH5, MUCL3, MX1, NA, NAIP, NBPF18P, NELFE, NFKB1, NFKB2, NFKBIA, NLRC3, NLRC4, NLRC5, NLRP1, NLRP10, NLRP11, NLRP12, NLRP13, NLRP14, NLRP2, NLRP3, NLRP4, NLRP5, NLRP6, NLRP7, NLRP8, NLRP9, NLRX1, NMI, NOD1, NOD2, NRCAM, OAS1, OAS2, OR10C1, OR12D2, OR14J1, OR2H1, OR2H2, OR2J1, OR2J2, OSMR, PARP9, PCDH15, PDCD6IPP2, PDIA3, PGLYRP4, PHF1, PHOSPH01; PKR, PMAIP1, PML, POMC, PPL, PPP1R11, PPP2R5A, PPP6R3, PPT2, PRR3, PRR9, PRRC2A, PRRT1, PSMB8, PSMB8-AS1, PSMB9, PSORS1C1, PSORS1C2, RAB40C, RALYL, REC8, REL, RELA, RELB, REPD TNFRSF8, RIG-1, RING1, RIP, RNAse L, RNF43, RNF5, RNF5P1, RPL13A, RPS18, RPS6KC1, RTP4, RXRB, S100A13, S100A7, S100A8, SAMD9L, SCNN1A, SECTM1, SEPX1, SERPINB7, SERPING1, SHMT1, SKIV2L, SLC44A4, SLCO5A1, SMURF2P1-LRRC37BP1, SNAPC3, SNHG32, SNORD32B, SNX14, SOCS1, SP110, SPRR1A, SPRR1B, SPRR3, SPRR4, SPTLC3, SSTR2, STAT1, STAT4, STOML1, SUN1, SYNGAP1, TAP1, TAP2, TAPBP, TBC1D5, TBK1, TCF19, TGFβ, THRB, TK2, TLR1, TLR10, TLR2, TLR3, TLR4, TLR5, TLR6, TLR7, TLR8, TLR9, TMEM140, TMEM185A, TMEM62, TNF, TNFR1, TNFR2, TNFRSF14, TNFRSF14-AS1, TNFSF10, TNFSF13B, TP53, TPTE2P5, TRA, TRADD, TRAF2, TRAF3, TRB, TRD, TRG, TRIM10, TRIM15, TRIM22, TRIM25, TRIM27, TRIM33, TRIM37, TRIM39, TRIM40, TRIT1, TRPM4, TSBP1, TSBP1-AS1, T TSEN15, UBB, UBE2K, UNG, USPS, VAMPS, VARS2, VEZT, VPS52, WARS, XAF1, XPOT, YY1AP1, ZC3HAV1, ZDHHC6, ZMYND11, ZNF248, ZNF512, ZNF610, and ZNRD1ASP.

59. The array of claim 56, wherein the plurality of oligonucleotide probes comprises at least one sequence selected from the group consisting of SEQ ID NOS:1-7,831.

60. The array of claim 56, wherein the plurality of oligonucleotide probes comprises at least one sequence selected from the group consisting of SEQ ID NOS:42, 48, 49, 56, 60, 152, 153, 154, 155, 156, 160, 161, 170, 174, 175, 176, 192, 195, 196, 205, 206, 207, 208, 209, 210, 211, 217, 219, 220, 221, 222, 235, 255, 294, 295, 298, 299, 300, 310, 315, 322, 329, 330, 331, 337, 602, 607, 608, 668, 669, 677, 678, 738, 750, 751, 756, 757, 761, 762, 769, 770, 773, 776, 777, 779, 829, 830, 842, 843, 846, 847, 855, 856, 857, 858, 860, 864, 869, 870, 877, 882, 904, 905, 916, 922, 923, 924, 925, 933, 942, 943, 959, 964, 965, 966, 969, 981, 982, 999, 1000, 1002, 1003, 1004, 1005, 1035, 1036, 1046, 1047, 1048, 1049, 1062, 1063, 1090, 1095, 1096, 1097, 1122, 1123, 1138, 1145, 1146, 1155, 1156, 1158, 1165, 1173, 1174, 1180, 1181, 1185, 1210, 1211, 1216, 1217, 1219, 1220, 1225, 1237, 1238, 1247, 1248, 1249, 1250, 1254, 1255, 1256, 1259, 1260, 1263, 1270, 1271, 1296, 1297, 1308, 1309, 1312, 1314, 1318, 1319, 1326, 1328, 1329, 1342, 1348, 1405, 1406, 1413, 1414, 1415, 1416, 1433, 1434, 1443, 1444, 1465, 1470, 1476, 1485, 1486, 1487, 1508, 1509, 1510, 1514, 1612, 1613, 1620, 1627, 1638, 1639, 1664, 1665, 1666, 1669, 1670, 1676, 1677, 1684, 1685, 1701, 1712, 1719, 1720, 1721, 1722, 1726, 1727, 1731, 1738, 1740, 1747, 1748, 1852, 1853, 1854, 1939, 2072, 2073, 2075, 2088, 2090, 2171, 2193, 2194, 2434, 2435, 2604, 2676, 2678, 2680, 2681, 2756, 2914, 2915, 2919, 2920, 3180, 3243, 3774, 3775, 3994, 3995, 3996, 3998, 3999, 4000, 4020, 4021, 4030, 4031, 4046, 4103, 4171, 4179, 4184, 4187, 4188, 4225, 4236, 4251, 4253, 4257, 4258, 4259, 4262, 4263, 4272, 4276, 4277, 4278, 4292, 4293, 4313, 4314, 4316, 4317, 4318, 4328, 4329, 4330, 4343, 4344, 4345, 4357, 4358, 4376, 4377, 4384, 4389, 4390, 4402, 4408, 4409, 4410, 4411, 4414, 4415, 4416, 4418, 4419, 4421, 4422, 4426, 4427, 4430, 4439, 4453, 4454, 4456, 4457, 4458, 4479, 4487, 4488, 4491, 4492, 4493, 4494, 4500, 4501, 4518, 4519, 4525, 4526, 4539, 4540, 4555, 4562, 4563, 4564, 4584, 4585, 4586, 4589, 4594, 4595, 4596, 4597, 4615, 4617, 4618, 4619, 4620, 4621, 4622, 4627, 4628, 4629, 4630, 4631, 4632, 4657, 4658, 4661, 4662, 4671, 4673, 4689, 4690, 4691, 4697, 4698, 4716, 4717, 4726, 4727, 4728, 4729, 4731, 4747, 4768, 4773, 4774, 4778, 4779, 4780, 4781, 4782, 4783, 4784, 4785, 4968, 4969, 4976, 4977, 4987, 4993, 4994, 4995, 4999, 5005, 5006, 5020, 5025, 5026, 5027, 5035, 5049, 5050, 5055, 5056, 5158, 5164, 5171, 5172, 5173, 5188, 5189, 5190, 5191, 5192, 5193, 5204, 5206, 5208, 5209, 5210, 5211, 5212, 5213, 5214, 5217, 5219, 5220, 5225, 5232, 5233, 5234, 5235, 5238, 5239, 5240, 5241, 5294, 5295, 5296, 5313, 5316, 5327, 5370, 5375, 5376, 5377, 5378, 5379, 5385, 5507, 5508, 5509, 5510, 5511, 5512, 5513, 5514, 5515, 5516, 5517, 5561, 5572, 5573, 5574, 5577, 5578, 5579, 5585, 5586, 5592, 5645, 5646, 5649, 5650, 5656, 5657, 5667, 5672, 5681, 5684, 5695, 5696, 5697, 5698, 5699, 5700, 5701, 5702, 5710, 5711, 5720, 5725, 5728, 5729, 5730, 5743, 5744, 5745, 5748, 5749, 5759, 5760, 5761, 5765, 5766, 5768, 5769, 5772, 5780, 5781, 5782, 5802, 5803, 5804, 5807, 5808, 5809, 5813, 5814, 5816, 5817, 5828, 5829, 5833, 5910, 5914, 5917, 5937, 5941, 5942, 5944, 5945, 5948, 5949, 5963, 6018, 6019, 6023, 6031, 6032, 6033, 6039, 6040, 6043, 6045, 6109, 6110, 6111, 6112, 6113, 6116, 6127, 6128, 6133, 6134, 6137, 6225, 6236, 6242, 6449, 6450, 6451, 6452, 6453, 6454, 6455, 6457, 6458, 6461, 6462, 6463, 6466, 6469, 6470, 6471, 6480, 6481, 6545, 6546, 6547, 6613, 6684, 6685, 6692, 6693, 6694, 6695, 6710, 6711, 6731, 6732, 6741, 6787, 6788, 6805, 6806, 6807, 6828, 6829, 6830, 6831, 6832, 6835, 6836, 6846, 6847, 6848, 6849, 6850, 6862, 6868, 6869, 6870, 6871, 6878, 6879, 6897, 6898, 6899, 6900, 6908, 6909, 6914, 6938, 6939, 6949, 6950, 6951, 6952, 6959, 6960, 6971, 6972, 6973, 6974, 6976, 6979, 7110, 7111, 7112, 7113, 7117, 7118, 7120, 7122, 7124, 7135, 7184, 7185, 7401, 7402, 7404, 7408, 7441, 7442, 7482, 7490, 7491, 7497, 7500, 7503, 7504, 7513, 7514, 7515, 7525, 7526, 7621, 7622, 7623, 7624, 7625, 7626, 7627, 7638, 7649, 7650, 7651, 7652, 7665, 7694, 7704, 7705, 7708, 7716, 7717, 7718, 7726, 7727, 7728, 7729, 7738, 7739, 7740, 7741, 7742, 7743, 7744, 7746, 7747, 7749, 7757, 7770, 7771, 7774, 7775, 7777, 7783, 7788, 7789, 7790, 7791, 7798, 7799, 7803, 7804, 7815, 7816, 7823, 7824, and 7825.

61. (canceled)
